(19) United States
(12) Patent Application Publication
(10) Pub. No.: US 2018/0116993 A1
(43) Pub. Date: May 3, 2018

(54) MODULATORS OF FARNESOID X RECEPTOR AND METHODS FOR THE USE THEREOF

(71) Applicant: XIAMEN UNIVERSITY, Xiamen (CN)

(72) Inventors: Yong LI, Pittsburgh, PA (US); Lihua JIN, Xiamen (CN); Weili ZHENG, Xiamen (CN); Yanlin ZHU, Xiamen (CN); Fusheng GUO, Xiamen (CN)

(73) Assignee: XIAMEN UNIVERSITY, Xiamen (CN)

(21) Appl. No.: 15/544,410

(22) PCT Filed: Jan. 21, 2016
(86) PCT No.: PCT/CN2016/071561

§ 371 Date: Jul. 18, 2017

(30) Foreign Application Priority Data
Jan. 22, 2015 (CN) 201510031454.X
May 6, 2015 (CN) 201510224709.4
May 6, 2015 (CN) 201510224817.1
May 6, 2015 (CN) 201510224818.6
May 22, 2015 (CN) 201510263191.5

(51) Int. Cl. A61K 31/235; A61K 31/4412; A61K 31/245; A61K 31/366; A61K 31/4409

(52) U.S. Cl. CPC A61K 31/235; A61K 31/4412; A61K 31/4409; A61K 31/366; A61K 31/245

(57) ABSTRACT

Compounds, compositions and methods are provided for treating the FXR-mediated disease or process in a mammal, comprising administering to the mammal a therapeutically effective amount of a compound claimed, wherein the FXR-mediated disease or condition linked to chronic liver diseases such as nonalcoholic fatty liver disease and nonalcoholic steatohepatitis; gastrointestinal diseases; cardiovascular diseases; metabolic diseases such as diabetes and obesity; inflammation, or cancer etc.

### CROSS-REFERENCE TO RELATED APPLICATION

This application claims priority to, and the benefit of, China patent application 201510263191.5, filed on May 22, 2015, which is a continuation of China patent application 201510224818.6, filed on May 6, 2015, which is a continuation of China patent application 201510224817.1, filed on May 6, 2015, which is a continuation of China patent application 201510224709.4, filed on May 6, 2015, which is a continuation of China patent application 201510031454.X, filed on Jan. 22, 2015, the disclosures of each of which are incorporated herein by reference.

### FIELD OF THE INVENTION

The present invention relates to compounds, compositions and methods for treating diseases or conditions mediated by farnesoid X receptor (FXR), and methods for the design and optimization of derivatives.

### BACKGROUND OF THE INVENTION

Nuclear receptors are a type of ligand-regulated transcription factors involved in a variety of biological processes (see, e.g., Mangelsdorf (1995) Cell 83, 835-839). For example, farnesoid X receptor (FXR), highly expressed in mammalian liver, intestine, kidney and adrenal gland, is one of 48 known human nuclear receptors. Nuclear receptors, such as FXR, play an important role in regulating virtually all aspects of human physiology including metabolism, inflammation, hepatic protection and regeneration, bile salt, fat and glucose homeostasis and other related physiological functions, by forming obligate heterodimers with RXR (retinoid X receptor) (see, e.g., Jin et al., (2010) Advanced drug delivery reviews 62, 1218-1226). As such, FXR has become an excellent drug target for the treatment of many FXR-mediated diseases like cancer, aging, metabolic diseases such as high blood glucose, insulin resistance, hypertriglyceridemia, hypercholesterolemia, diabetes, obesity, biliary obstruction, gallstones, nonalcoholic fatty liver, atherosclerosis and other diseases (see, e.g., Fiorucci et al., (2010) Current Medicinal Chemistry, 17, 139-159 and Carotti et al., (2014) Current Topics in Medicinal Chemistry, 14, 2129-2142).

Small molecules known as ligands play important roles in modulating the activity of nuclear receptors, since the binding of ligands can induce the conformational changes that determine the recruitment of coregulators. The coregulators include coactivators like the p160 factors also referred to as the steroid receptor coactivators (SRC) family, and corepressors such as SMART (silencing mediator for retinoid and thyroid hormone receptors) and N-CoR (nuclear corepressor). As such, the functions of nuclear receptors are tightly associated with their cognate ligands. Given the critical roles of these ligands in human disease, they have been studied intensively in pharmaceutical development.

Cholestasis is composed of a variety of human liver diseases such as primary biliary cirrhosis, primary sclerosing cholangitis, cystic fibrosis, and intrahepatic cholestasis of pregnancy (see, e.g., Pellicciari et al., (2002) Journal of medicinal chemistry 45, 3569-3572). In the liver, activation of FXR induces transcription of transporter genes involved in promoting bile acid clearance and represses genes involved in bile acid synthesis. The enterohepatic circulation of bile acids enables the absorption of fats and fat-soluble vitamins from the intestine and allows the elimination of cholesterol, toxins, and metabolic by-products such as bilirubin from the liver. FXR-null mice exhibit cholestatic liver disorder. In the bile duct-ligation and α-naphthylisothiocyanate models of cholestasis, GW4064 treatment resulted reductions in inflammation, other markers of liver damage and increased expression of genes involved in bile acid transport (see e.g., Liu et al., (2003) Journal of Clinical Investigation 112, 1678-1687). These suggest therapeutic applications of FXR ligands can been used to treat liver disorders associated with cholestasis. FXR agonists may be useful in the treatment of cholestatic liver disease, such as cholestasis, liver inflammation, liver damage, primary sclerosing cholangitis, cystic fibrosis, and intrahepatic cholestasis of pregnancy.

FXR modulators impacts both bile acid synthesis and lipid metabolism, resulting of them to be effective pharmaceutical agent in preventing and treating liver diseases associated with bile acid mediated cellular injury, fatty liver disease, liver cancer as well as atherosclerosis and cardiovascular disease. Moreover, studies on wide type and FXR^{-/-} mice have determined that FXR play pleotropic roles in regulating triglyceride, lipid, cholesterol, glucose metabolism in addition to bile acids homeostasis (see e.g., Sinal et al., (2000) Cell 102, 731-44). Hypertriglycerides are a predictor of coronary heart disease risk factor, strategies targeting the hypertriglyceride is a well prevention and treatment for coronary heart disease risk (see e.g., Cullen (2000) The American Journal of Cardiology 86, 943-949). This is mainly attributed to the inverse relationship between serum triglycerides (TGs) and HDL cholesterol, since low levels of HDL increase the risk of vascular disease. Bile acid lowers serum TGs, reducing SREBP-1c and lipogenic genes dependent on activating FXR and inducing the expression of SHP (see e.g., Lambert et al., (2003) The Journal of biological chemistry 278, 2563-2570 and Watanabe et al., (2004) The Journal of clinical investigation 113, 1408-1418). As such, FXR modulators can be used to treat or prevent the hypertriglyceride and the related coronary heart disease.

FXR-null mice show features in glucose tolerance and Muslin resistance (see e.g., Zhang et al., (2006) Proceedings of the National Academy of Sciences of the United States of America 103, 1006-1011). In addition to GW4064, FXR ligand Ivemectin was recently found to be specifically regulating glucose and cholesterol homeostasis dependent on FXR (see e.g., Jin et al., (2013) Nature communications 4, 1937). As such, FXR is a drug target in treating or preventing hyperglycemia, hypercholesterol, obesity, diabetes as well as disorders related to glucose and cholesterol metabolism.

FXR is an ideal target for nonalcoholic fatty liver disease (NAFLD) drug development due to its crucial roles in lipid metabolism (see e.g., Carr and Reid, (2015) Curr Atheroscler Rep 17, 500). Activation of FXR reduced liver expression of genes involved in fatty acid synthesis, lipogenesis, and gluconeogenesis, as well as reducing the steatosis of obese rat (see e.g., Cipriani et al., (2010) J Lipid Res 51, 771-784). FXR ligands Avermectin analogues are effective in regulating metabolic parameters tested, including reducing hepatic lipid accumulation, lowering serum cholesterol and glucose levels, and improving NAFLD in a FXR dependent manner (see e.g., Jin et al., (2015) Scientific reports 5, 17288). Taken together, FXR has been proposed as a target for improving non-alcoholic steatohepatitis (NASH), or non-alcoholic fatty liver disease (NAFLD) from steatosis to cirrhosis, and even liver cancer.

Hypercholesterolemia and dyslipidemia is are important risk factor for cardiovascular disease (CVD) and atherosclerosis, characterized by elevated plasma triglycerides (TGs) and low HDL-cholesterol (HDL-C), in combination with obesity, elevated blood glucose levels, and/or hypertension termed the metabolic syndrome (see e.g., Porez et al., (2012) J Lipid Res 53, 1723-1737). FXR activation protects against atherosclerosis development as well as hyperlipidemia in ApoE−/− mice (see e.g., Hartman et al., (2009) J Lipid Res 50, 1090-1100 and Mencarelli et al., (2009) Am J Physiol Heart Circ Physiol 296, H272-281). Thus, FXR ligands might be used in prevention and treatment of atherosclerosis and cardiovascular disease.

FXR inhibits inflammation through antagonizing NF-kappaB pathway (see e.g., Wang et al., (2008) Hepatology 48, 1632-1643). FXR deficiency is susceptible to gallbaldder inflammation and cholesterol gallstone disease (CGD), indicating that FXR is a potential target in treating CGD (see e.g., Moschetta et al., (2004) Nature medicine 10, 1352-1358). Emerging roles for FXR in the gut include protection against bacterial overgrowth and maintenance of intestinal barrier function. FXR activation protects against murine models of induced colitis (see e.g., Gadaleta et al., (2011) Gut 60, 463-472 and Vavassori et al., (2009) J Immunol 183, 6251-6261). Theses suggest that FXR modulators can be useful as a therapeutic strategy for inflammation, such as inflammatory bowel disease.

FXR activation by increased bile acid flux is a signal for liver regeneration in mice. FXR may promote homeostasis not only by regulating expression of appropriate metabolic target genes but also by driving homeotrophic liver growth (see e.g., Huang et al., (2006) Science 312, 233-6). However, irregular regeneration of hepatocytes with cells over proliferation has been reported as an important factor in carcinogenesis (see e.g., Ueno et al., (2001) Hepatology 33, 357-362 and Wang et al., (2008) Hepatology 48, 1632-1643). FXR^{-/-} mice spontaneously developed liver tumors, while intestinal-selective FXR modulators activation is sufficient to prevent hepatic malignancy (see, e.g., Yang et al., (2007) Cancer Res 67, 863-867 and Degirolamo et al., (2015) Hepatology 61:161-70). FXR deficiency in the intestine promotes Wnt signaling with expansion of the basal proliferative compartment, while FXR activation can induce the apoptosis of colon cancer cells (see e.g., Modica et al., (2008) Cancer Res 68, 9589-9594). Taken together, FXR can be a target to protect against carcinogenesis such as liver and intestinal cancer.

FXR also plays a critical role in aging-induced fatty liver (see e.g., Xiong et al., (2014) J Hepatol. 60(4):847-54), and expression and activity of FXR is increased in the livers of the long-lived Little mice, both suggesting an association between FXR and aging (see e.g., Jiang et al., (2013) Mech Ageing Dev. 134(9): 407-15). Activation of FXR is able to alleviate age-related liver regeneration defects (see e.g., Chen et al., (2010) Hepatology 51(3):953-62). These findings highlight FXR as a potential target of drug design for disorders related to aging such as liver regeneration and extension of chronological lifespan.

The regulation of FXR by ligands has beneficial effects on bone metabolism through modulating bone formation, differentiation and resorption, resulting in preventing bone loss and enhancing bone mass gain (see e.g., Cho et al., (2013) J Bone Miner Res. 28(10):2109-21), suggesting therapeutic roles of FXR ligands in treating disorders related to bone formation such as osteoporosis, bone hyperplasia and osteoarthritis.

Despite the considerable attention of FXR as a key regulator in human disease, the therapeutic potentials of FXR ligands remain to be further studied. The use of bile acids such as CA and CDCA is limited in humans because they bind into FXR with a low affinity and cause significant hepatotoxicity as well as increased LDL (see e.g., Watanabe et al., (2004) Journal of Clinical Investigation 113, 1408-1418). CDCA can also bind to ileal bile acid-binding protein (I-BABP), bile acid transporters and other proteins. Many synthetic FXR ligands have also been described, but have limitations owing to side effects and uncertain bioavailability (see e.g., Watanabe et al., (2011) The Journal of biological chemistry 286, 26913-26920). Accordingly, there is a need for compositions, compounds, and systems to treat FXR-mediated diseases.

### SUMMARY OF THE INVENTION

The present application relates to compounds, or pharmaceutically acceptable salt, isomers, or prodrugs thereof, that bind to the farnesoid X receptor (FXR), for the treatment of FXR-mediated diseases or conditions, including but not limited to inflammation, analgesia, cholestasis, colitis, chronic liver diseases, gastrointestinal diseases, renal diseases, cardiovascular disease, kidney disease, inflammatory disorder, metabolic diseases and various cancers.

Another aspect of this invention is directed to methods of treating, preventing, inhibiting, or ameliorating the symptoms of a disease or disorder or a condition that is modulated by FXR activity, by administering to the mammal a therapeutically effective amount of at least one compound or combinations of compounds disclosed herein.

In some embodiments, a FXR-mediated disease is selected from hyperglycemia, insulin resistance, hypertriglyceridemia, hypercholesterolemia, diabetes, obesity, metabolic syndrome, metabolic disorders and related diseases, diseases of the liver (hepatic disease), fatty liver disease (hepatic steatosis), non-alcoholic fatty liver disease (NAFLD), steatohepatitis, non-alcoholic steatohepatitis (NASH), cirrhosis, fibrosis, chronic and acute liver failure, biliary cirrhosis, primary sclerosing cholangitis, cholestasia, gallstone atherosclerosis, inflammation, cancer, and combinations thereof.

As used herein, the term “FXR ligand” refers to any compounds that regulate FXR activity as full agonists, partial agonists, antagonists, inverse agonists, or selective nuclear receptor modulators, due to their diverse characteristics in FXR binding mode, regulating transcription and post-translational modification and their ability in inducing FXR to recruit various co-regulators. Post-translational modifications, such as SUMOylation and phosphorylation, are also differentially associated with transactivation or transrepression, respectively.

As used herein, the term “FXR activity” refers any FXR activities relating to therapeutic effects in human disease. For example, FXR activity regulated by compounds for use in accordance with the present invention include, but is not limited to, transcriptional activity, phosphorylation, acetylation, methylation, ubiquitination, sumoylation, any other posttranscriptional activity, any other protein modification, and protein-protein interactions relating to signal transduction.

In some embodiments, the compounds for use in the methods described herein may be formulated as a therapeutically effective amount of pharmaceutical compositions. Pharmaceutical compositions of this invention may comprise the compounds described herein or a pharmaceutically acceptable salt thereof and a pharmaceutically acceptable carrier. Such compositions may optionally comprise an additional therapeutic agent.

As used herein, “EC50” refers to a dosage, concentration or amount of a said compound which induces a response halfway between the baseline and maximum after a specified exposure time, commonly used as a measure of drug's potency.

The technology used herein, is also described in Jin et al., (2013) Nature communications 4, 1937 and Jin et al., (2015) Scientific reports 5, 17288.

### BRIEF DESCRIPTION OF DRAWINGS

FIG. 1. The H&E staining of liver sections in mice with APAP-induced liver injury.

FIG. 2. The H&E staining (A &C) and Oil Red O staining (B) of liver sections from db/db mice treated with 10 mg/kg of compounds for 11 days.

FIG. 3. The Oil Red O staining of liver sections. A, illustrates KK-Ay mice treated with 20 mg/kg of tschimganine illustrated in Table 9. B illustrates wild type C57B6/J mice treated with 10 mg/kg of tschimganidine once daily for 10 days. The mice were fed with high-fat diet for 2 months before the experiments. C illustrates mice explained in Table 10. D illustrates mice explained in Table 12.

FIG. 4 Masson's staining and Sirius red staining of liver sections. A, Masson's staining of liver sections from mice illustrated in FIG. 2A. B, Masson's staining of liver sections from db/db mice treated with 10 mg/kg of tschimganine for 7 days. C, illustrate the Masson's staining of liver sections from mice illustrated in FIG. 3D. D, Sirius red staining of liver sections from mice illustrated in FIG. 2A.

### EXAMPLES

The following specific examples:

### Example 1. Compounds Modulate FXR Activity

In search of novel ligands for FXR, we used FXR ligand binding domain (LBD) as a bait to screen chemical libraries based on AlphaScreen biochemical assay, which determines the efficacy of small molecules in influencing binding affinity of FXR with coregulator peptides (see e.g., Jin et al., (2013) Nature communications 4, 1937). Results from commercial available compound library revealed Feroline, Tschimganidine, Tschimganine, Tschimgine, Ferutinin, Juniferdin Derivative,9, Hedragonic acid, other compounds listed in Table 1 potently promoted the interaction of FXR with coactivator LXXLL motifs from SRC1-2 and SRC2-3 in a concentration dependent manner (Table 1), indicating these compounds are able to regulate FXR activity. Notably, their synthetic derivatives or analogues showed similar results (Table 2).

To confirm these compounds in regulating FXR activity in cells, cell-based reporter assay was employed to characterize the transcriptional properties of FXR in response to the compounds. COS-7 cells were cotransfected with plasmids encoding full length FXR. In agreement with Alphascreen results, these compounds significantly transactivated FXR using an EcRE reporter (Table 1 & 2), indicating that they can interact with FXR and modulate FXR activity.

The binding of the cofactor peptide motifs to FXR ligand binding domain (LBD) in response to ligands was determined by AlphaScreen assays using a hexahistidine detection kit from Perkins-Elmer. The FXR LBD protein was purified as described previously (see e.g., Jin et al., (2013) Nature communications 4, 1937). The experiments were conducted with approximately 20-40 nM receptor LBD and 20 nM biotinylated cofactor peptides in the presence of 5 μg/ml donor and acceptor beads in a buffer containing 50 mM MOPS, 50 mM NaF, 0.05 mM CHAPS, and 0.1 mg/ml bovine serum albumin, all adjusted to a pH of 7.4. The peptides with an N-terminal biotinylation are listed below: SRC1-2, SPSSHSSLTERHKILHRLLQEGSP; SRC2-3, QEPVSPKKKENALLRYLLDKDDTKD.

For transient transfection assay, COS-7 cells were maintained in DMEM containing 10% fetal bovine serum and were transiently transfected using Lipofectamine 2000 (Invitrogen). Before 24 h of transfection, 24-well plates were plated. The cells were co-transfected with plasmids encoding full-length FXR and the cognate luciferase reporter EcRE-Luc. Ligands were added 5 hours after transfection. Cells were harvested 24 hours later for the luciferase assays according to the manufacturer's instructions (Dual-Luciferase® Reporter Assay System, Promega). Luciferase activities were normalized to renilla activity co-transfected as an internal control.

TABLE 1
Potency of compounds in regulating FXR activity as determined by the ability in inducing FXR to recruit coregulator
motif by AlphaScreen assay and transactivation of FXR reporter activity in COS-7 cells cotransfected with plasmids
encoding full-length FXR and an EcRE luciferase response reporter. The indicated values are EC50 (μM) measured.

| | | Cell-based FXR | | EC₅₀ (μM) of | |
|---|---|---|---|---|---|
| | | transactivation | | FXR binding by | |
| | | Fold | | AlphaScreen | |
| | | Induction | | assay | |
| | | by 5 μM | EC₅₀ | recruit | recruit |
| Compounds | Structures | compound | (μM) | SRC2-3 | SRC1-2 |
| Feroline | 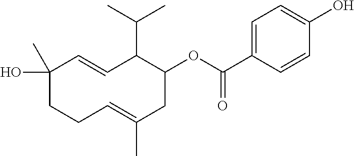 [SMILES: C/C1=C\CCC(C)(O)/C=C/C(C(C)C)C(OC(=O)c2ccc(O)cc2)C1] | >10 | <1 μM | <1 μM | <1 μM |
| Tschimganidine | 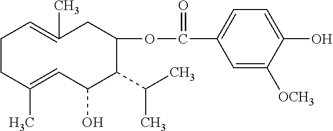 [SMILES: COc1cc(C(=O)OC2C/C(C)=C/CC/C(C)=C/[C@@H](O)[C@H]2C(C)C)ccc1O] | >10 | <1 μM | <1 μM | <1 μM |
| Tschimganine | 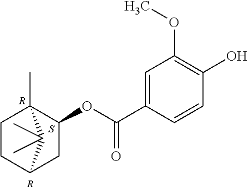 | >10 | <1 μM | <1 μM | <1 μM |
| Tschimgine | 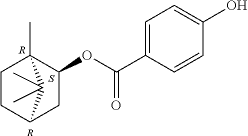 | >10 | <1 μM | <1 μM | <1 μM |
| Ferutinin | 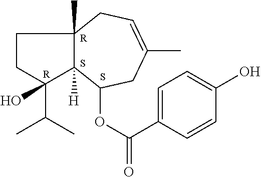 | 5 | 1-10 μM | 1-10 μM | 1-10 μM |
| Juniferdin Derivative,9 (XD-10) | 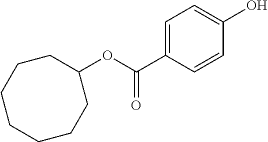 [SMILES: O=C(OC1CCCCCCC1)c1ccc(O)cc1] | 8 | <1 μM | <1 μM | <1 μM |
| Hedragonic acid | 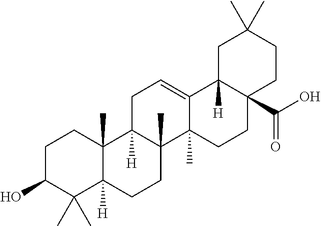 | 5 | 1-10 μM | 1-10 μM | 1-10 μM |
| Tricyclo- [3.3.1.1^{3,7}]decane- 1-carboxylic acid, 3-chloro-, 4- methoxyphenyl ester | 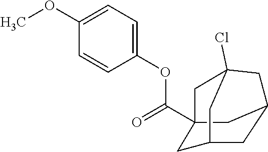 [SMILES: COc1ccc(OC(=O)C23CC4CC(CC(Cl)(C4)C2)C3)cc1] | >10 | <1 μM | <1 μM | <1 μM |
| Tricyclo- [3.3.1.1^{3,7}]decane- 1-methanol, 1-(3- aminobenzoate) | 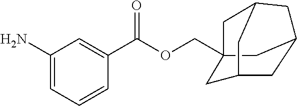 [SMILES: Nc1cccc(C(=O)OCC23CC4CC(CC(C4)C2)C3)c1] | >10 | <1 μM | <1 μM | <1 μM |
| Tricyclo- [3.3.1.1^{3,7}]decane- 1-carboxylic acid, 4-hydroxy- phenyl ester | 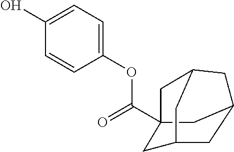 [SMILES: O=C(Oc1ccc(O)cc1)C12CC3CC(CC(C3)C1)C2] | >10 | <1 μM | <1 μM | <1 μM |

TABLE 2
Potency of synthesized compounds in regulating FXR activity as
determined by the ability in inducing FXR to recruit coregulator
binding motif by AlphaScreen assay and transactivation of FXR
reporter activity in COS-7 cells cotransfected with plasmids
encoding full-length FXR and an EcRE luciferase response
reporter. The indicated values are fold changes (positive:
induction; minus: repression) and EC50 (μM) measured.
N/D means not determined.

| | Cell-based FXR | | | |
|---|---|---|---|---|
| | transactivation | | EC₅₀ (μM) of FXR binding | |
| | Fold changes | | by AlphaScreen assay | |
| | by 5 μM | | recruit | |
| Compounds | compound | EC₅₀ (μM) | SRC2-3 | recruit SRC1-2 |
| XD-0 | 5 | 1-10 μM | 1-10 μM | 1-10 μM |
| XD-1 | 5 | 1-10 μM | 1-10 μM | 1-10 μM |
| XD-3 | 5 | 1-10 μM | 1-10 μM | 1-10 μM |
| XD-4 | >10 | <1 μM | <1 μM | <1 μM |
| XD-5 | >10 | <1 μM | <1 μM | <1 μM |
| XD-6 | >10 | 1-10 μM | 1-10 μM | 1-10 μM |
| XD-8 | 4 | 1-10 μM | 1-10 μM | 1-10 μM |
| XD-9 | 4 | 1-10 μM | 1-10 μM | 1-10 μM |
| XD-11 | 4 | 1-10 μM | 1-10 μM | 1-10 μM |
| XD-12 | 1.5 | N/D | >10 μM | >10 μM |
| XD-13 | 4 | <1 μM | <1 μM | <1 μM |
| XD-14 | 3 | 1-10 μM | N/D | N/D |
| XD-15 | 3 | 1-10 μM | N/D | N/D |
| XD-16 | 5 | <1 μM | <1 μM | <1 μM |
| XD-17 | 3 | <1 μM | <1 μM | <1 μM |
| XD-18 | 3 | <1 μM | <1 μM | <1 μM |
| XD-19 | 1.5 | N/D | N/D | N/D |
| XD-20 | 6 | 1-10 μM | 1-10 μM | 1-10 μM |
| XD-21 | 1.5 | N/D | N/D | N/D |
| XD-22 | 3 | >1 μM | 1-10 μM | 1-10 μM |
| XD-23 | −30 | <1 μM | <0.5 μM | <0.5 μM |
| XD-24 | 1.2 | <1 μM | N/D | N/D |
| XD-25 | 1.5 | <1 μM | N/D | N/D |
| XD-26 | −2 | <1 μM | N/D | N/D |
| XD-28 | 3 | <1 μM | <0.5 μM | <0.5 μM |
| XD-29 | 3 | <1 μM | <0.5 μM | <0.5 μM |
| XD-30 | 3 | <1 μM | <0.5 μM | <0.5 μM |
| XD-31 | −5 | <1 μM | <1 μM | <1 μM |
| XD-32 | −5 | <1 μM | <1 μM | <1 μM |
| XD-33 | 2 | 1-10 μM | <1 μM | <1 μM |
| XD-34 | 2 | 1-10 μM | <1 μM | <1 μM |
| XD-35 | 2 | 1-10 μM | 1-10 μM | N/D |
| XD-36 | 3 | 1-10 μM | 1-10 μM | N/D |

### Example 2. Preparation of Compounds

The following examples illustrate synthetic routes of compounds listed in Table 3 with chemical structures and ¹H-NMR data for compounds disclosed herein. The rest compounds are commercial available.

### XD-0

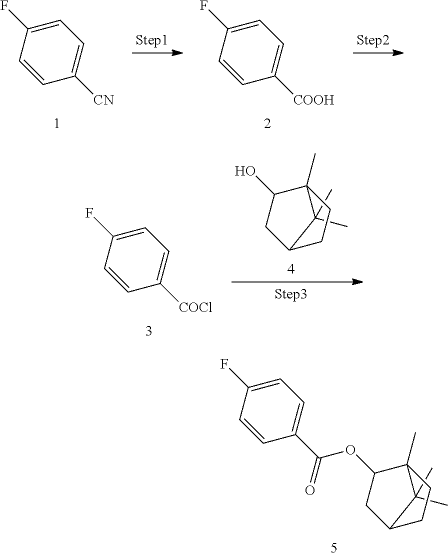 [SMILES: N#Cc1ccc(F)cc1 ; O=C(O)c1ccc(F)cc1 ; O=C(Cl)c1ccc(F)cc1 ; CC1(C)C2CCC1(C)C(O)C2 ; CC1(C)C2CCC1(C)C(OC(=O)c1ccc(F)cc1)C2]

Add compound 1 and 70% sulfuric acid solution, stirring and heating reflux react for 24 h. When completed, adjust to pH of 3 using 2 M NaOH in ice bath, and then extract twice with dichloromethane. Combine the organic phase, wash with water and saturated sodium chloride solution. Dry the reaction under anhydrous sodium sulfate, remove the drier by filtration, and obtain the compound 2 by concentration. Add dimethyl sulfoxide to compound 2, stir and heating reflux for 1 h, then cool down to room temperature, remove the solvent under reduced pressure to obtain the compound 3. Add dry dichloromethane 15 ml to the compound 3, followed by adding dry trimethylamine, cool down to 0-10° C., then add compound 4 to the reaction, continue to stir for 24 h with TLC monitoring.

### XD-1 and XD-3

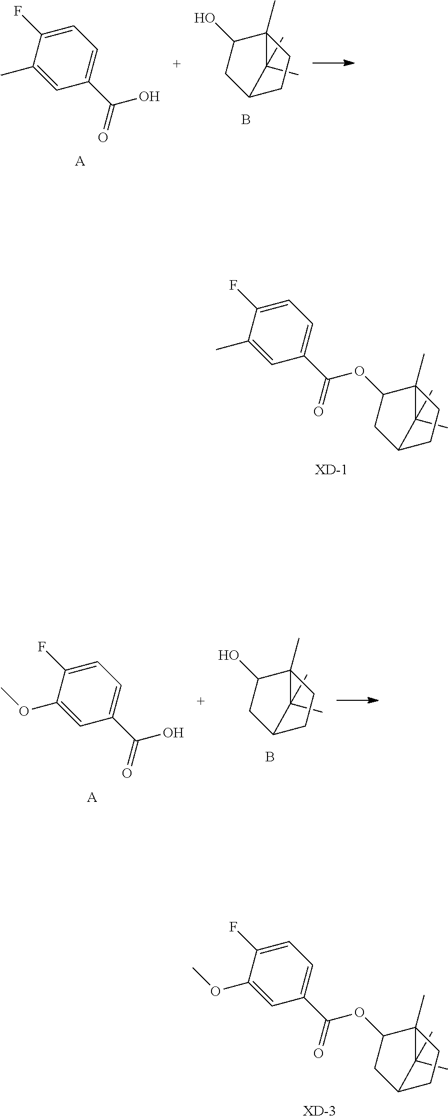

The synthesis operation of XD-1 is the same as that of XD-3. Add compound A, 4-dimethylaminopyridine, p-toluenesulfonic acid, then dichloromethane into the flask, stir for 5 min in ice bath, add dicyclohexylcarbodiimide, stir for another 5 min in ice bath, then add compound B, put the system at room temperature to react for 24 h with TLC monitoring.

### XD-15 and XD-4

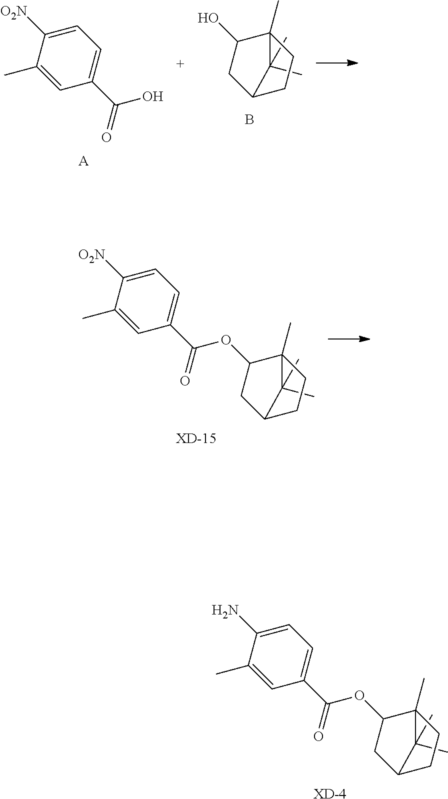

The synthesis operation of XD-15 is the same as that of XD-3. Mix XD-15 250 mg and 5% Pd/C 50 mg into a round-bottomed flask, then add methanol 5 ml into the system, replace the air in the system with hydrogen for 3 times, react for 18 h at room temperature for hydrogenation with TLC monitoring. Filtration when the reaction is completed, the solvent was concentrated under reduced pressure, XD-4, a kind of light yellowish brown solid, was obtained by column chromatography.

### XD-14 and XD-5

The synthesis strategy of XD-14 is the same as that of XD-3, and the operation of XD-5 is the same as that of XD-4.

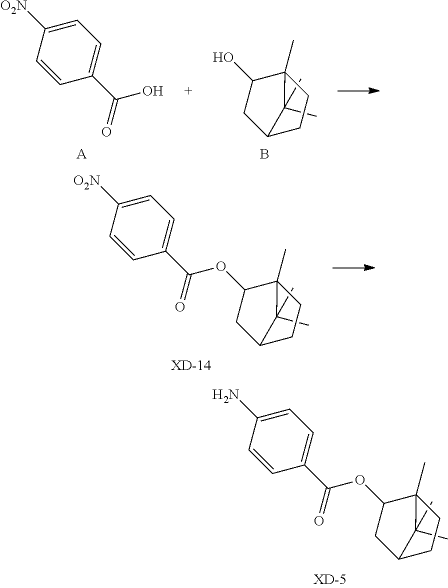

### XD-6

The synthesis strategy of XD-6 is the same as that of XD-3.

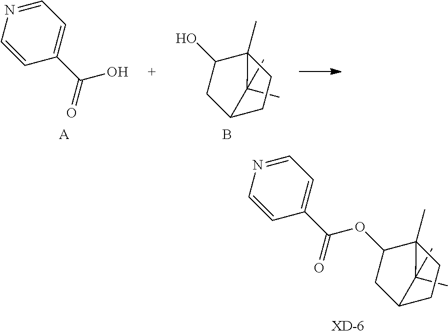

### XD-8

The synthesis strategy of XD-8 is the same as that of XD-3.

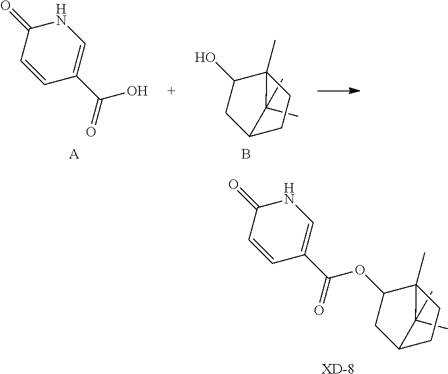

### XD-9

The synthesis strategy of XD-9 is the same as that of XD-3.

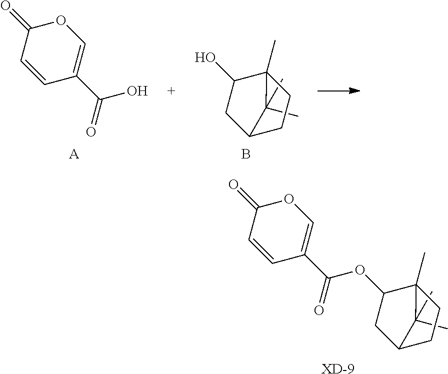

### XD-10

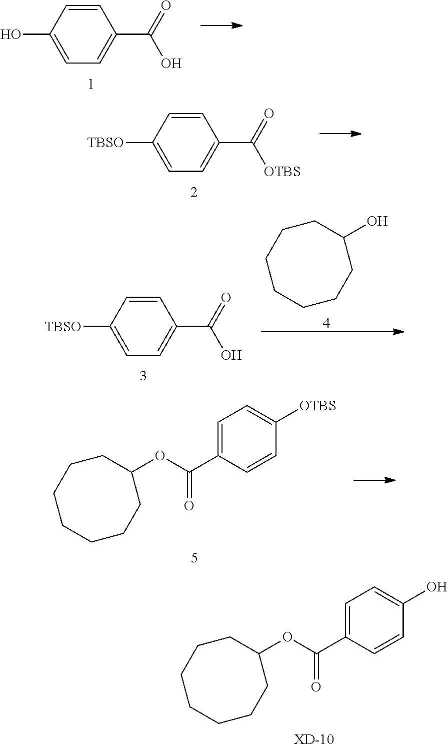

Add compound 1, t-butyl dimethyl chlorosilane, then ethyl acetate into the system, stir in cold water bath. Then add 8.1 g trimethylamine, put the system at room temperature to react for 1 h. Remove triethylamine hydrochloride by filtration when the reaction is completed. The filtrate was allowed to stand for 1 h, and then for filtration once again to remove triethylamine hydrochloride. The solvent was concentrated under reduced pressure to give compound 2.

Add 8 g of compound 2 and tetrahydrofuran 120 ml, then add 5% NaOH 15 ml dropwisely, and continue to stir for 15 min. When the reaction is completed, evaporate out most of the tetrahydrofuran by reducing pressure at 40° C., then add water 80 ml, stir in ice bath, adjust to pH3-pH4 using 1 M HCl to separate out white solid, suction filtration, washing with water, the solid was naturally dried, compound 3, a kind of white solid, was obtained by column chromatography.

Add compound 3 750 mg, 4-dimethylaminopyridine 75 mg and p-toluenesulfonic acid 105 mg into a flask, then add dichloromethane 15 ml, stir for 5 min in ice bath. Then add dicyclohexylcarbodiimide 930 mg, stir for another 5 min in ice bath, add compound 4 380 mg, put the system at room temperature to react for 20 h with TLC monitoring. Perform suction filtration when the reaction is completed, the solvent was removed under reduced pressure and compound 5 was obtained by column chromatography.

Add compound 5 280 mg, tetrahydrofuran 5 ml and tetrabutylammonium fluoride trihydrate 30 mg into a reaction bottle, stir for 10 h at room temperature with TLC monitoring. When the reaction is completed, remove the solvent under reducing pressure. White solid XD-10 was obtained by column chromatography.

### XD-11

The synthesis strategy of XD-11 is the same as that of XD-10.

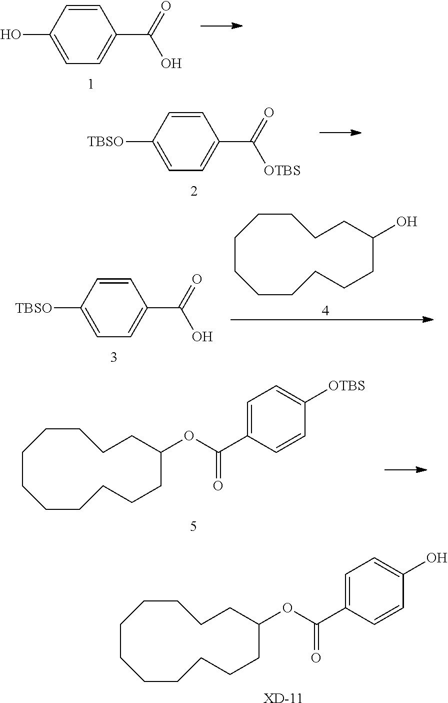

### XD-12

The synthesis strategy of compound C and XD-12 are the same as that of XD-3 and XD-4,

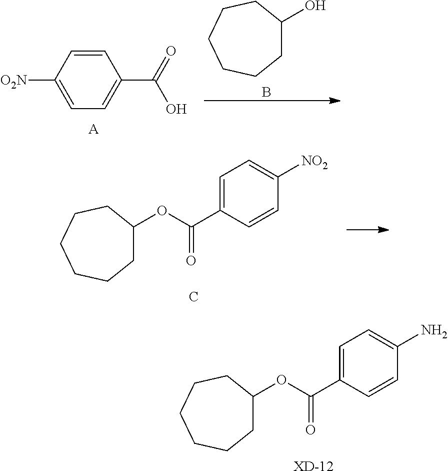

### XD-13

The synthesis strategy of XD-13 is the same as that of XD-3.

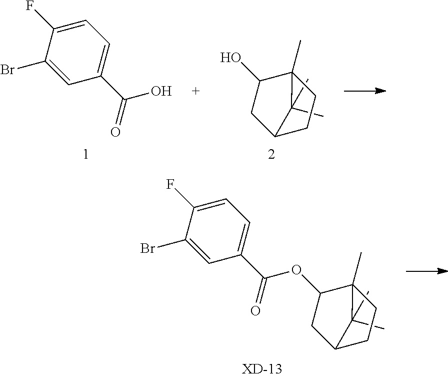

### XD-24 and XD-16

The synthesis operation of XD-24 and XD-16 are the same as that of XD-3 and XD-4, respectively.

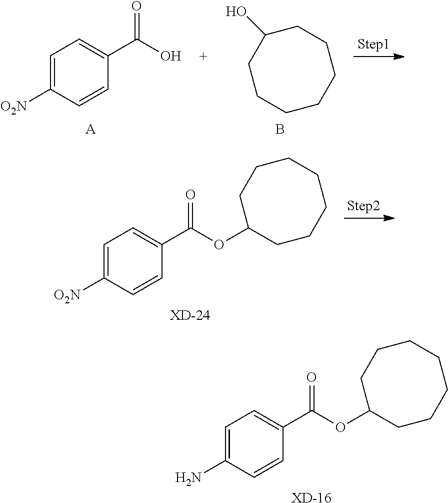

### XD-17

The synthesis operation of XD-17 is the same as that of XD-10.

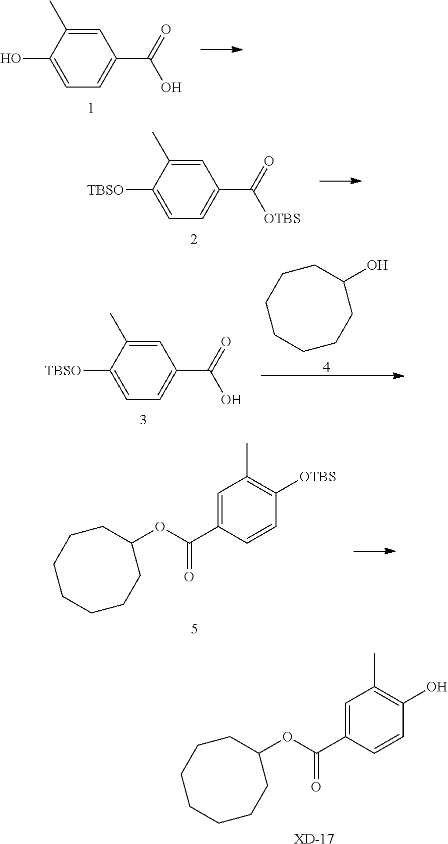

### XD-18

The synthesis strategy of XD-18 is the same as that of XD-10.

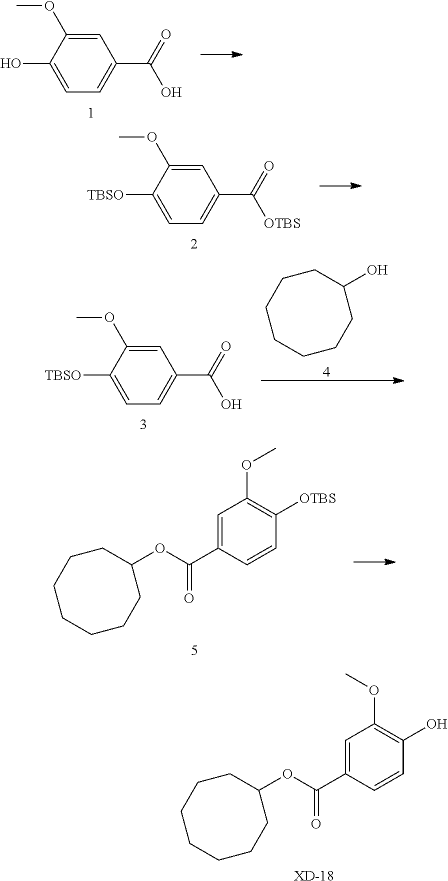

### XD-19

The synthesis strategy of XD-19 is the same as that of XD-10.

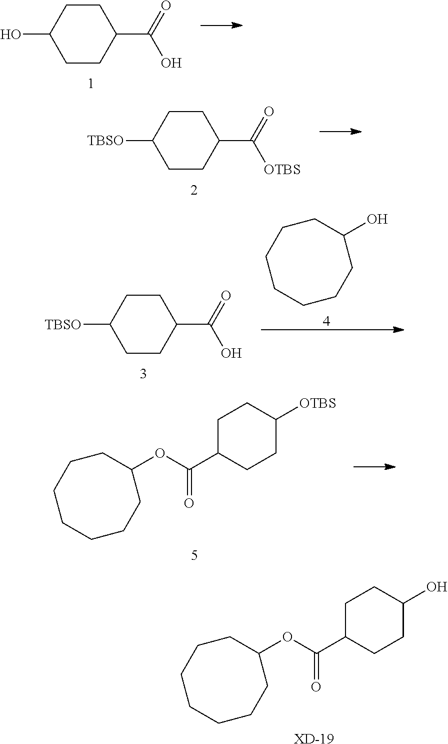

### XD-20

The synthesis operation of XD-20 is the same as that of XD-10.

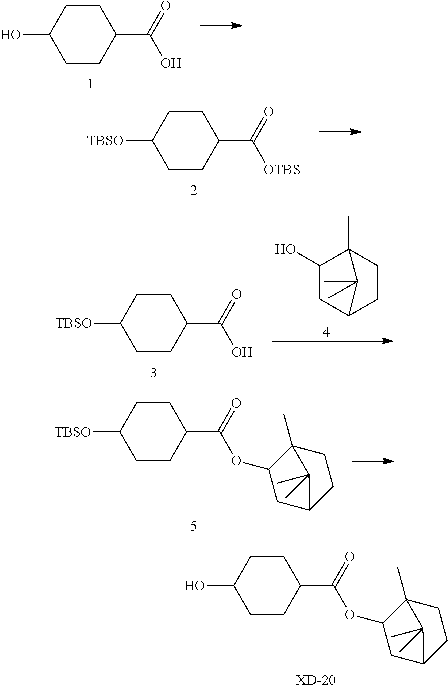

### XD-21

The synthesis strategy of XD-21 is the same as that of XD-4.

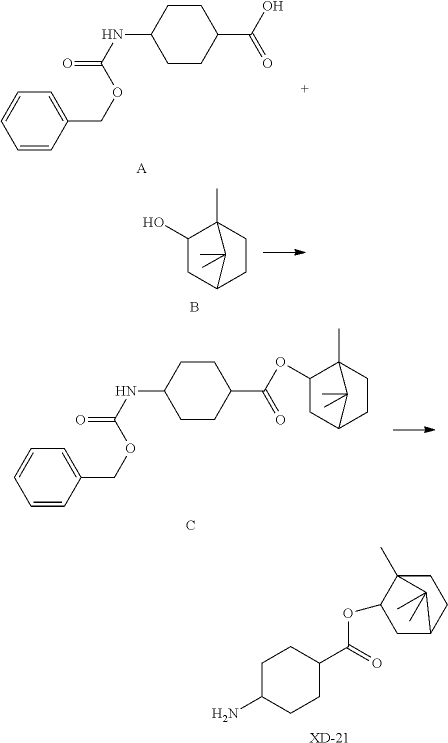

### XD-25 and XD-22

The synthesis strategies of XD-25 and XD-22 are the same as that of XD-3 and XD-4,

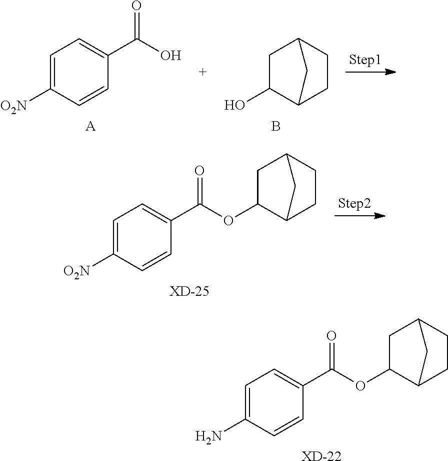

### XD-26 and XD-23

The synthesis strategies of XD-26 and XD-23 are the same as those of XD-3 and XD-4, respectively.

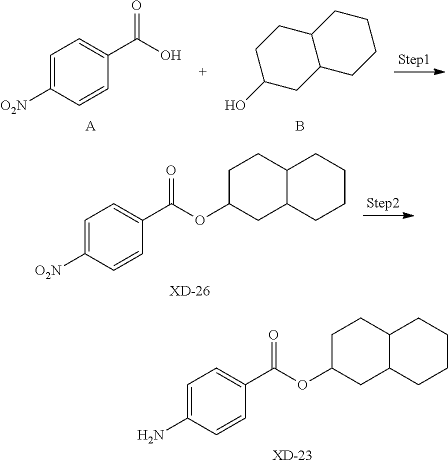

### XD-24

The synthesis operation of XD-24 is the same as that of XD-3.

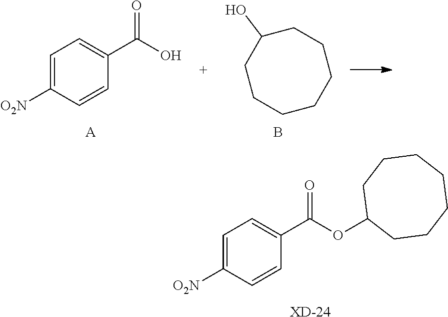

### XD-28

As the reaction below, the synthesis operation of XD-28 is the same as that of XD-10.

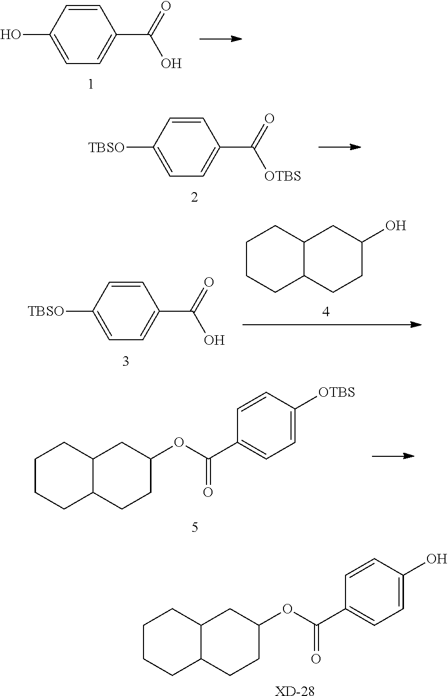

### XD-29

As the following reaction, the synthesis operation of XD-29 is the same as that of XD-10.

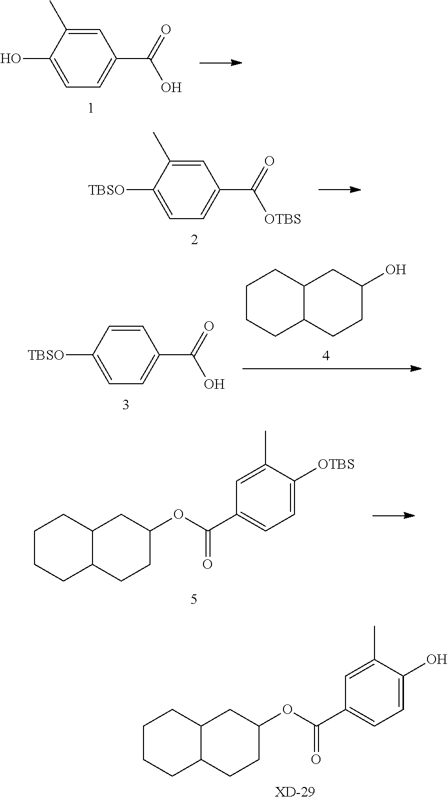

### XD-30

As the following reaction, the synthesis operation of XD-30 is the same as that of XD-10.

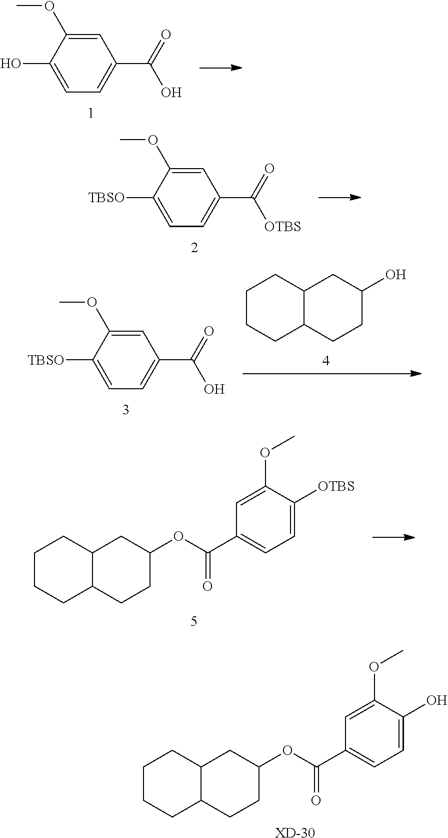

### XD-35 and XD-31

As the following reaction, the synthesis operation of XD-35 is the same as that of XD-3, and the synthesis operation of XD-31 is the same as that of XD-4.

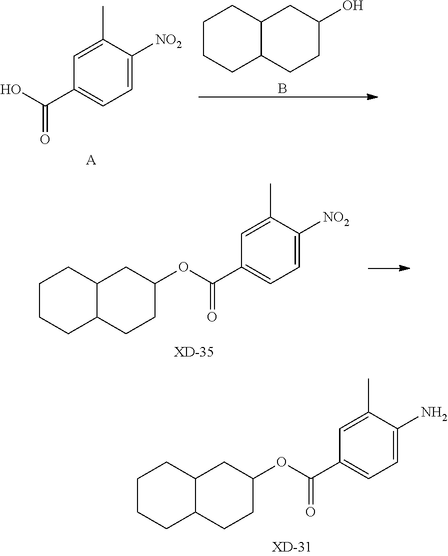

### XD-36 and XD-32

As the following reaction, the synthesis operation of XD-36 is the same as that of XD-3, and the synthesis operation of XD-32 is the same as that of XD-4.

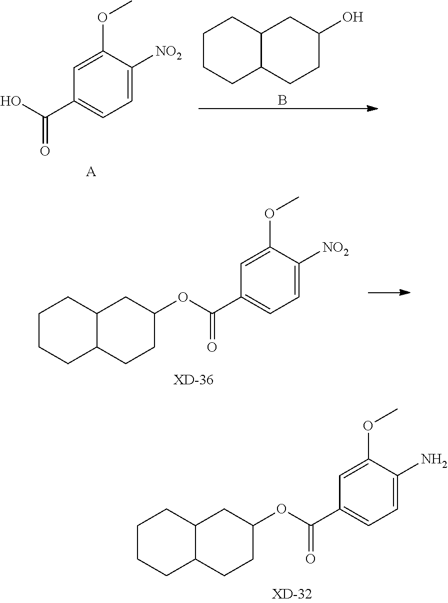

### XD-33

As the following reaction, the synthesis operation of XD-33 is the same as that of XD-3.

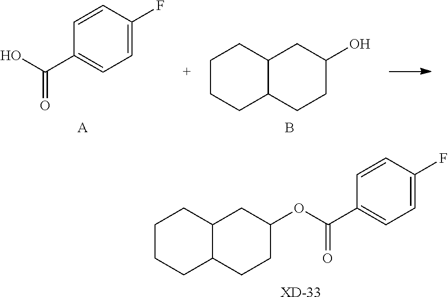

### XD-34

As the following reaction, the synthesis operation of XD-34 is the same as that of XD-3.

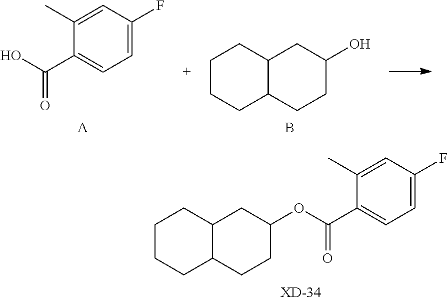

### XD-35

As the following reaction, the synthesis operation of XD-35 is the same as that of XD-3.

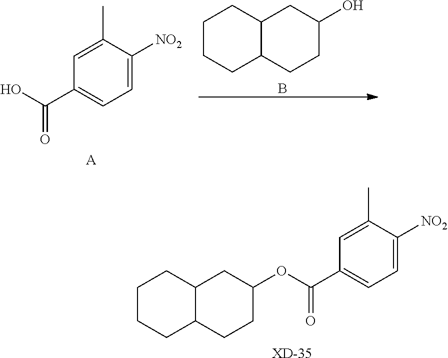

### XD-36

As the following reaction, the synthesis operation of XD-36 is the same as that of XD-3.

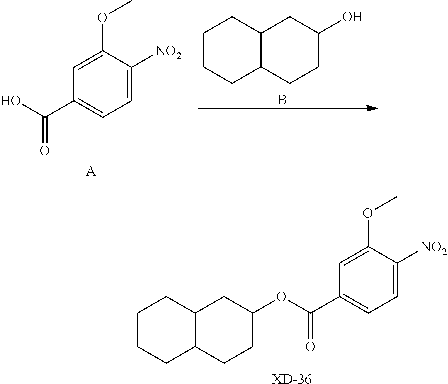

TABLE 3
Chemical No., names, structures and ¹H-NMR data for compounds disclosed herein.

| No. | Full Name | Structure | ¹H-NMR data |
|---|---|---|---|
| XD-0 | 1,7,7-trimethyl- bicyclo[2.2.1]- heptan-2-yl 4-fluorobenzoate | 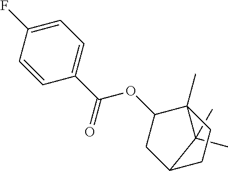 [SMILES: CC1(C)C2CCC1(C)C(OC(=O)c1ccc(F)cc1)C2] | ¹H-NMR (400 MHz, DMSO, ppm) δ 8.10-7.90 (m, 2H), 7.41-7.34 (m, 2H), 5.10-4.80 (m, 1H), 2.40-1.10 (m, 7H), 1.10-0.80 (m, 9H). |
| XD-1 | 1,7,7-trimethyl- bicyclo[2.2.1]- heptan-2-yl 4-fluoro-3- methylbenzoate | 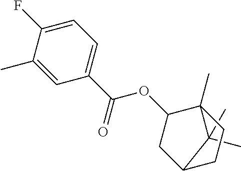 | ¹H-NMR (400 MHz, DMSO, ppm) δ 7.95-7.78 (m, 2H), 7.35-7.25 (m, 1H), 5.10-4.75 (m, 1H), 2.45-1.10 (m, 10H), 1.10-0.80 (m, 9H). |
| XD-3 | 1,7,7-trimethyl- bicyclo[2.2.1]- heptan-2-yl 4-fluoro-3- methoxybenzoate | 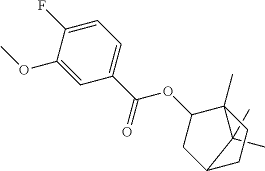 | ¹H-NMR (400 MHz, DMSO, ppm) δ 7.70-7.50 (m, 2H), 7.45-7.35 (m, 1H), 5.10-4.80 (m, 1H), 4.00-3.85 (m, 3H), 2.45-1.10 (m, 7H), 1.10- 0.80 (m, 9H). |
| XD-4 | 1,7,7-trimethyl- bicyclo[2.2.1]- heptan-2-yl 4-amino-3- methylbenzoate | 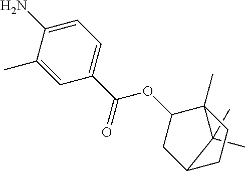 | ¹H-NMR (400 MHz, DMSO, ppm) δ 7.75-7.45 (m, 2H), 7.00-6.55 (m, 1H), 5.10-4.65 (m, 1H), 2.45-1.10 (m, 10H), 1.10-0.80 (m, 9H). |
| XD-5 | 1,7,7-trimethyl- bicyclo[2.2.1]- heptan-2-yl 4-aminobenzoate | 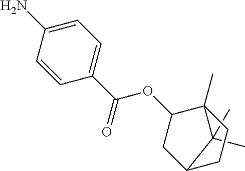 [SMILES: CC1(C)C2CCC1(C)C(OC(=O)c1ccc(N)cc1)C2] | ¹H-NMR (300 MHz, DMSO, ppm) δ 7.80-7.60 (m, 2H), 6.80-6.60 (m, 2H), 5.00-4.70 (m, 1H), 2.45-1.10 (m, 7H), 1.10-0.80(m, 9H). |
| XD-6 | 1,7,7-trimethyl- bicyclo[2.2.1]- heptan-2-yl isonicotinate | 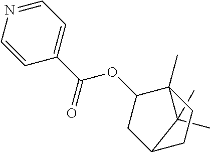 [SMILES: CC1(C)C2CCC1(C)C(OC(=O)c1ccncc1)C2] | ¹H-NMR (400 MHz, DMSO, ppm) δ 8.85-8.75 (m, 2H), 7.90-7.70 (m, 2H), 5.15-4.75 (m, 1H), 2.45-1.13 (m, 7H), 1.13-0.73 (m, 9H). |
| XD-8 | 1,7,7-trimethyl- bicyclo[2.2.1]- heptan-2-yl 6-oxo-1,6- dihydropyridine- 3-carboxylate | 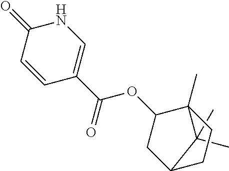 [SMILES: CC1(C)C2CCC1(C)C(OC(=O)c1ccc(=O)[nH]c1)C2] | ¹H-NMR (400 MHz, DMSO, ppm) δ 12.11 (br, 1H), 8.10-7.90 (m, 1H), 7.88-7.74 (m, 1H), 6.40 (d, J = 9.61, 1H), 5.05-4.70 (m, 1H), 2.40-1.08 (m, 7H), 1.08-0.80 (m, 9H). |
| XD-9 | 1,7,7-trimethyl- bicyclo[2.2.1]- heptan-2-yl 6-oxo-6H-pyran- 3-carboxylate | 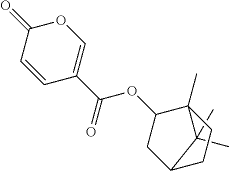 [SMILES: CC1(C)C2CCC1(C)C(OC(=O)c1ccc(=O)oc1)C2] | ¹H-NMR (400 MHz, DMSO, ppm) δ 8.75-8.45 (m, 1H), 7.95-7.75 (m, 1H), 6.55-6.40 (m, 1H), 5.05-4.70 (m, 1H), 2.40-1.10 (m, 7H), 1.10- 0.75 (m, 9H). |
| XD-10 | cyclooctyl 4- hydroxybenzoate | 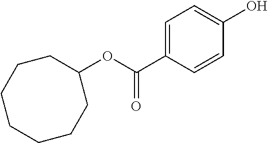 [SMILES: O=C(OC1CCCCCCC1)c1ccc(O)cc1] | ¹H-NMR (400 MHz, DMSO, ppm) δ 10.28 (br, 1H), 7.78 (d, J = 8.65, 2H), 6.83 (d, J = 8.66, 2H), 5.07- 4.95 (m, 1H), 1.90-1.30 (m, 14H). |
| XD-11 | cyclododecyl 4- hydroxybenzoate | 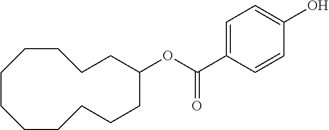 [SMILES: O=C(OC1CCCCCCCCCCC1)c1ccc(O)cc1] | ¹H-NMR (300 MHz, DMSO, ppm) δ 10.30 (s, 1H), 7.79 (d, J = 8.73, 2H), 6.84 (d, J = 8.74, 2H), 5.20- 5.04 (m, 1H), 1.85-1.65 (m, 2H), 1.65-1.47 (m, 2H), 1.47-1.10 (m, 18H). |
| XD-12 | cycloheptyl 4-aminobenzoate | 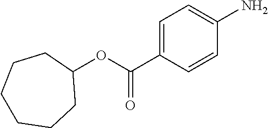 [SMILES: Nc1ccc(C(=O)OC2CCCCCC2)cc1] | ¹H-NMR (400 MHz, DMSO, ppm) δ 7.75-7.60 (m, 2H), 6.80-6.60 (m, 2H), 5.05-4.95 (m, 1H), 1.95-1.35 (m, 12H). |
| XD-13 | 1,7,7-trimethyl- bicyclo[2.2.1]- heptan-2-yl 3- bromo-4-fluoro- benzoate | 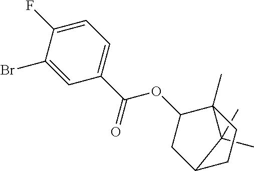 [SMILES: CC1(C)C2CCC1(C)C(OC(=O)c1ccc(F)c(Br)c1)C2] | ¹H-NMR (300 MHz, DMSO, ppm) δ 8.25-8.10 (m, 1H), 8.07-7.92 (m, 1H), 7.62-7.50 (m, 1H), 5.10-4.75 (m, 1H), 2.45-1.15 (m, 7H), 1.15- 0.80 (m, 9H). |
| XD-14 | 1,7,7-trimethyl- bicyclo[2.2.1]- heptan-2-yl 4-nitrobenzoate | 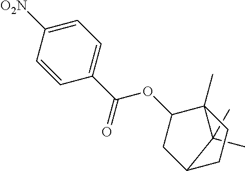 [SMILES: CC1(C)C2CCC1(C)C(OC(=O)c1ccc([N+](=O)[O-])cc1)C2] | ¹H-NMR (300 MHz, DMSO, ppm) δ 8.37 (d, J = 8.64, 2H), 8.22 (d, J = 8.74, 1H), 8.15 (d, J = 8.73, 1H), 5.15-4.80 (m, 1H), 2.50-1.15 (m, 7H), 1.15-0.80 (m, 9H). |
| XD-15 | 1,7,7-trimethyl- bicyclo[2.2.1]- heptan-2-yl 3-methyl-4-nitro- benzoate | 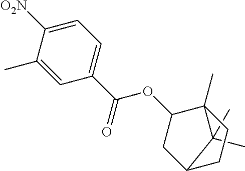 | ¹H-NMR (300 MHz, DMSO, ppm) δ 8.15-7.90 (m, 3H), 5.12-4.80 (m, 1H), 2.61-2.53 (m, 3H), 2.48-1.15 (m, 7H), 1.15-0.80 (m, 9H). |
| XD-16 | cyclooctyl 4-aminobenzoate | 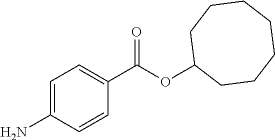 [SMILES: Nc1ccc(C(=O)OC2CCCCCCC2)cc1] | ¹H-NMR (300 MHz, MeOD, ppm) δ 8.14 (d, J = 8.41, 2H), 7.47 (d, J = 8.46, 2H), 5.28-5.13 (m, 1H), 2.05- 1.5 (m, 14H). |
| XD-17 | cyclooctyl 4-hydroxy-3- methylbenzoate | 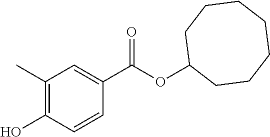 [SMILES: Cc1cc(C(=O)OC2CCCCCCC2)ccc1O] | ¹H-NMR (300 MHz, CDCl3, ppm) δ 7.83(s, 1H), 7.79 (dd, J = 8.32, 1.95, 1H), 6.79 (d, J = 8.32, 1H), 5.42 (s, 1H), 5.23-5.12 (m, 1H), 2.28 (s, 3H), 1.98-1.49 (m, 14H). |
| XD-18 | cyclooctyl 4-hydroxy-3- methoxybenzoate | 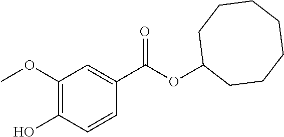 [SMILES: COc1cc(C(=O)OC2CCCCCCC2)ccc1O] | ¹H-NMR (400 MHz, CDCl3, ppm) δ 7.63(dd, J = 8.30, 1.87, 1H), 7.55 (d, J = 1.86, 1H), 6.93 (d, J = 8.31, 1H), 5.98 (s, 1H), 5.21-5.12 (m, 1H), 3.95 (s, 3H), 1.98-1.48 (m, 14H). |
| XD-19 | cyclooctyl 4-hydroxy- cyclohexane- carboxylate | 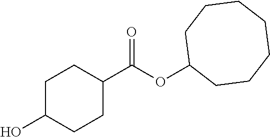 [SMILES: O=C(OC1CCCCCCC1)C1CCC(O)CC1] | ¹H-NMR (400 MHz, CDCl3, ppm) δ 4.90-4.85 (m, 1H), 4.08-3.88 (m, 1H), 3.68-3.55 (br, 1H), 2.35-2.15 (m, 1H), 2.10-1.95 (m, 4H), 1.80- 1.64 (m, 6H), 1.60-1.42 (m, 10H), 1.35-1.23 (m, 2H). |
| XD-20 | 1,7,7-trimethyl- bicyclo[2.2.1]- heptan-2-yl 4-hydroxy- cyclohexane- carboxylate | 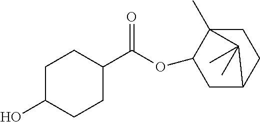 [SMILES: CC1(C)C2CCC1(C)C(OC(=O)C1CCC(O)CC1)C2] | ¹H-NMR (400 MHz, CDCl3, ppm) δ 4.93-4.85 and 4.70-4.60 (m, 1H), 4.10-3.85 and 3.53-3.40 (m, 1H), 3.70-3.55 (br, 1H), 2.40-2.15 (m, 2H), 2.14-1.97 (m, 4H), 1.97-1.87 (m, 2H), 1.87-1.05 (m, 9H), 1.05- 0.75 (m, 9H). |
| XD-21 | 1,7,7-trimethyl- bicyclo[2.2.1]- heptan-2-yl 4-aminocyclo- hexane- carboxylate | 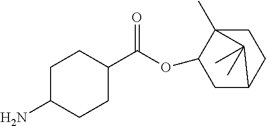 [SMILES: CC1(C)C2CCC1(C)C(OC(=O)C1CCC(N)CC1)C2] | ¹H-NMR (300 MHz, MeOD, ppm) δ 4.90-4.65 (m, 1H), 4.62 (br, 2H), 3.17-3.00 (m, 1H), 2.47-1.92 (m, 6H), 1.90-1.05 (m, 9H), 1.05-0.80 (m, 9H). |
| XD-22 | bicyclo[2.2.1]- heptan-2-yl 4-aminobenzoate | 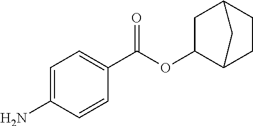 [SMILES: Nc1ccc(C(=O)OC2CC3CCC2C3)cc1] | ¹H-NMR (300 MHz, MeOD, ppm) δ 8.12 (dt, J = 8.73, 2.25, 2H), 7.44 (dt, J = 8.72, 2.19, 2H), 4.90-4.80 (m, 1H), 2.50-2.32 (m, 2H), 1.95- 1.80 (m, 1H), 1.75-1.45 (m, 4H), 1.40-1.12 (m, 3H). |
| XD-23 | decahydro- naphthalen-2-yl 4-aminobenzoate | 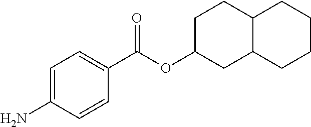 [SMILES: Nc1ccc(C(=O)OC2CCC3CCCCC3C2)cc1] | ¹H-NMR (400 MHz, MeOD, ppm) δ 8.17-8.05 (m, 2H), 7.42-7.32 (m, 2H), 5.27-4.85 (m, 1H), 2.20-0.90 (m, 16H). |
| XD-24 | cyclooctyl 4-nitrobenzoate | 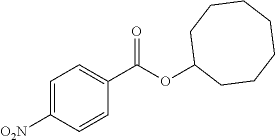 [SMILES: O=C(OC1CCCCCCC1)c1ccc([N+](=O)[O-])cc1] | ¹H-NMR (400 MHz, CDCl3, ppm) δ 8.28 (dt, J = 8.93, 2.04, 2H), 8.20 (dt, J = 8.95, 1.96, 2H), 5.28-5.20 (m, 1H), 2.05-1.85 (m, 4H), 1.85- 1.72 (m, 2H), 1.72-1.47 (m, 8H). |
| XD-25 | bicyclo[2.2.1]- heptan-2-yl 4-nitrobenzoate | 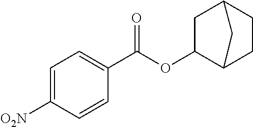 [SMILES: O=C(OC1CC2CCC1C2)c1ccc([N+](=O)[O-])cc1] | ¹H-NMR (400 MHz, CDCl3, ppm) δ 8.28(dt, J = 8.98, 2.10), 8.19 (dt, J = 9.00, 2.01), 4.90 (d, J = 7.02, 1H), 2.47 (d, J = 4.66, 1H), 2.38 (s, 1H), 1.87 (m, 1H), 1.70-1.56 (m, 3H), 1.56-1.46 (m, 1H), 1.32-1.12 (m, 3H). |
| XD-26 | decahydro- naphthalen-2-yl 4-nitrobenzoate | 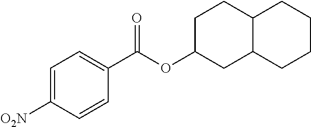 [SMILES: O=C(OC1CCC2CCCCC2C1)c1ccc([N+](=O)[O-])cc1] | ¹H-NMR (400 MHz, CDCl3, ppm) δ 8.32-8.25 (m, 2H), 8.24-8.17 (m, 2H), 5.35-4.95 (m, 1H), 2.25-0.80 (m, 16H). |
| XD-28 | decahydro- naphthalen-2-yl 4-hydroxy- benzoate | 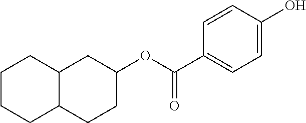 [SMILES: O=C(OC1CCC2CCCCC2C1)c1ccc(O)cc1] | ¹H-NMR (300 MHz, DMSO, ppm) δ 10.30 (s, 1H), 7.90-7.70 (m, 2H), 6.95-6.75 (m, 2H), 5.15-4.70 (m, 1H), 2.10-0.75 (m, 16H). |
| XD-29 | decahydro- naphthalen-2-yl 4-hydroxy-3- methylbenzoate | 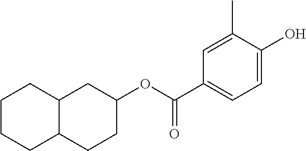 [SMILES: Cc1cc(C(=O)OC2CCC3CCCCC3C2)ccc1O] | ¹H-NMR (300 MHz, DMSO, ppm) δ 10.22 (s, 1H), 7.75-7.55 (m, 2H), 6.95-6.80 (m, 1H), 5.15-4.70 (m, 1H), 2.14 (s, 3H), 2.05-0.80 (m, 16H). |
| XD-30 | decahydro- naphthalen-2-yl 4-hydroxy-3- methoxybenzoate | 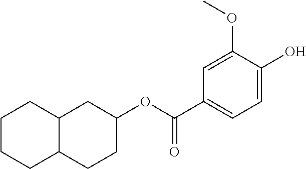 [SMILES: COc1cc(C(=O)OC2CCC3CCCCC3C2)ccc1O] | ¹H-NMR (400 MHz, CDCl3, ppm) δ 7.67-7.61 (m, 1H), 7.58-7.52 (m, 1H), 6.98-6.88 (m, 1H), 5.25-4.88 (m, 1H), 4.00-3.90 (m, 3H), 2.15- 0.80 (m, 16H). |
| XD-31 | decahydro- naphthalen-2-yl 4-amino-3- methylbenzoate | 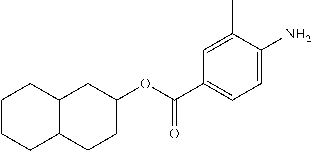 [SMILES: Cc1cc(C(=O)OC2CCC3CCCCC3C2)ccc1N] | ¹H-NMR (300 MHz, CDCl3, ppm) δ 8.05-7.85 (m, 2H), 7.70-7.55 (m, 1H), 5.30-4.85 (m, 1H), 2.62 (s, 3H), 2.15-0.80 (m, 16H). |
| XD-32 | decahydro- naphthalen-2-yl 4-amino-3- methoxybenzoate | 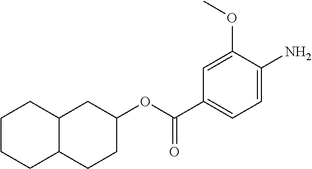 [SMILES: COc1cc(C(=O)OC2CCC3CCCCC3C2)ccc1N] | ¹H-NMR (400 MHz, CDCl3, ppm) δ 7.75-7.66 (m, 2H), 7.66-7.60 (m, 1H), 5.27-4.90 (m, 1H), 4.05-3.90 (m, 3H), 2.15-0.85 (m, 16H). |
| XD-33 | decahydro- naphthalen-2-yl 4-fluorobenzoate | 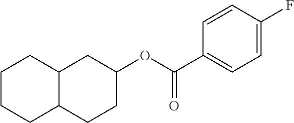 [SMILES: O=C(OC1CCC2CCCCC2C1)c1ccc(F)cc1] | ¹H-NMR (400 MHz, DMSO, ppm) δ 8.10-7.90 (m, 2H), 7.45-7.25 (m, 2H), 5.15-4.75 (m, 1H), 2.05-0.80 (m, 16H). |
| XD-34 | decahydro- naphthalen-2-yl 4-fluoro-3- methylbenzoate | 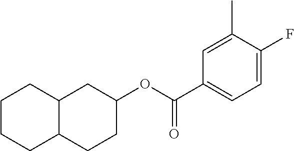 [SMILES: Cc1cc(C(=O)OC2CCC3CCCCC3C2)ccc1F] | ¹H-NMR (400 MHz, DMSO, ppm) δ 7.95-7.75 (m, 2H), 7.35-7.20 (m, 1H), 5.15-4.75 (m, 1H), 2.35-2.20 (m, 3H), 2.05-0.85 (m, 16H). |
| XD-35 | decahydro- naphthalen-2-yl 3-methyl-4-nitro- benzoate | 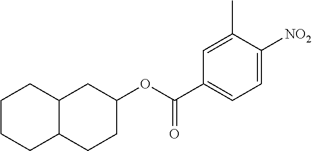 [SMILES: Cc1cc(C(=O)OC2CCC3CCCCC3C2)ccc1[N+](=O)[O-]] | ¹H-NMR (400 MHz, CDCl3, ppm) δ 8.05-7.91 (m, 3H), 5.30-4.91 (m, 1H), 2.70-2.60 (m, 3H), 2.15-0.85 (m, 16H). |
| XD-36 | decahydro- naphthalen-2-yl 3-methoxy-4- nitrobenzoate | 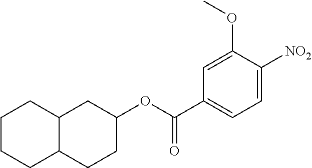 [SMILES: COc1cc(C(=O)OC2CCC3CCCCC3C2)ccc1[N+](=O)[O-]] | ¹H-NMR (300 MHz, CDCl3, ppm) δ 7.90-7.80 (m, 1H), 7.80-7.73 (m, 1H), 7.73-7.65 (m, 1H), 5.33-4.93 (m, 1H), 4.08-4.00 (m, 3H), 2.20- 0.80 (m, 16H). |

### Example 3. Inhibitory Effects on Cancer Cells

The MTT assay is a colorimetric assay for assessing cell metabolic activity. It is widely used in high-throughput screening of antitumor drugs due to its high sensitivity and economical features. In this example, MTT was used to detect the inhibitory effects of compounds on various cancer cell lines. Cancer cells including human colorectal adenocarcinoma HCT15 cells, RA-resistant human colon cancer HCT116 cells, human cervical carcinoma Hela cells, human hepatoma HepG2 cells and human breast cancer MCF7 cells were cultured in DMEM medium containing 10% fetal bovine serum. 20 uM or 40 uM of compounds were administered to treat cells, cell viability is detected by MTT assay and expressed as a percentage of the cells treated with DMSO (Table 4).

As shown in Table 4, for the detected cell lines, the cell viabilities were significantly lower with compounds treatment than that in the DMSO control cells, indicating the therapeutic effects of these compounds on human colon cancer including RA-resistant colon cancer, cervical cancer, liver cancer and breast cancer.

TABLE 4
The inhibition of proliferation of various cancer cells after 48 hours treatment with
different concentrations of compounds disclosed herein was assessed by MTT assay. Cell viability
is expressed as a percentage of the cells treated with DMSO.

| Compound | | | | | | | | | | |
|---|---|---|---|---|---|---|---|---|---|---|
| treatment | HCT15 | | HCT116 | | HepG2 | | Hela | | MCF-7 | |
| (48 h)/% | 20 uM | 40 uM | 20 uM | 40 uM | 20 uM | 40 uM | 20 uM | 40 uM | 20 uM | 40 uM |
| Feroline | 100 ± 2 | 76 ± 6^{a} | 100 ± 5^{a} | 80 ± 2^{a} | 84 ± 1^{a} | 65 ± 3^{c} | 83 ± 1.8^{c} | 67 ± 3^{c} | 92 ± 13 | 86 ± 12 |
| Tschimgan- | 85 ± 9 | 60 ± 10^{b} | 95 ± 4^{a} | 40 ± 7^{b} | 94 ± 1^{a} | 48 ± 4^{b} | 96 ± 4^{a} | 30 ± 5^{a} | 100 ± 4 | 85 ± 4^{b} |
| idine | | | | | | | | | | |
| Tschimganine | 100 ± 1 | 80 ± 9 | 97 ± 1 | 90 ± 4^{a} | 100 ± 1 | 97 ± 3^{a} | 98 ± 2 | 97 ± 4^{a} | 100 ± 3 | 99 ± 3 |
| Tschimgine | 97 ± 6 | 31 ± 2^{a} | 93 ± 4^{a} | 25 ± 5^{b} | 97 ± 2^{a} | 30 ± 3^{a} | 94 ± 3^{a} | 31 ± 4^{a} | 93 ± 2^{a} | 32 ± 2^{b} |
| XD-0 | 81 ± 4^{b} | 56 ± 2^{c} | 95 ± 4 | 62 ± 6^{c} | 81 ± 2 | 76 ± 7 | 90 ± 5^{a} | 71 ± 3^{a} | 100 ± 2 | 85 ± 4^{c} |
| XD-1 | 93 ± 2^{a} | 42 ± 3^{c} | 96 ± 2 | 62 ± 3^{c} | 100 ± 2 | 62 ± 7 | 100 ± 5 | 70 ± 3^{b} | 100 ± 2 | 65 ± 1^{c} |
| XD-3 | 73 ± 5^{b} | 40 ± 1^{c} | 80 ± 4^{b} | 41 ± 4^{c} | 100 ± 3 | 70 ± 3^{c} | 100 ± 1 | 66 ± 3^{c} | 100 ± 4 | 40 ± 2^{c} |
| XD-4 | 79 ± 2^{b} | 41 ± 3^{c} | 81 ± 2^{b} | 27 ± 1^{c} | 90 ± 5 | 42 ± 1^{c} | 95 ± 4 | 36 ± 2^{c} | 100 ± 9 | 23 ± 1^{c} |
| XD-5 | 80 ± 4^{b} | 30 ± 4^{c} | 88 ± 2^{a} | 30 ± 3^{c} | 95 ± 1 | 40 ± 1^{c} | 93 ± 4 | 46 ± 3^{c} | 100 ± 3 | 20 ± 1^{c} |
| XD-6 | 57 ± 2^{c} | 42 ± 3^{c} | 61 ± 1^{c} | 45 ± 2^{c} | 99 ± 1 | 70 ± 2^{c} | 100 ± 1 | 76 ± 1^{c} | 100 ± 8 | 61 ± 1^{c} |
| XD-8 | 78 ± 4^{b} | 60 ± 4^{c} | 92 ± 7^{a} | 60 ± 3^{c} | 90 ± 3^{a} | 64 ± 3 | 97 ± 3^{a} | 71 ± 3^{c} | 94 ± 10 | 75 ± 7^{c} |
| XD-9 | 49 ± 2^{c} | 42 ± 3^{c} | 58 ± 2^{c} | 25 ± 2^{c} | 90 ± 3 | 64 ± 3^{b} | 80 ± 3 | 59 ± 5 | 72 ± 6 | 31 ± 1^{c} |
| XD-10 | 60 ± 4^{c} | 41 ± 4^{c} | 92 ± 6 | 30 ± 4^{c} | 90 ± 3 | 35 ± 1^{c} | 90 ± 4 | 40 ± 3^{c} | 93 ± 7 | 28 ± 1^{c} |
| XD-11 | 89 ± 2^{b} | 78 ± 3^{b} | 92 ± 2^{a} | 70 ± 7^{b} | 100 ± 1 | 97 ± 1 | 100 ± 3 | 100 ± 2 | 100 ± 7 | 100 ± 2 |
| XD-12 | 65 ± 4^{c} | 54 ± 4^{c} | 77 ± 3^{b} | 65 ± 3^{c} | 100 ± 2 | 97 ± 3 | 100 ± 2 | 90 ± 5^{b} | 100 ± 4 | 82 ± 9^{b} |
| XD-13 | 100 ± 2 | 71 ± 3^{b} | 100 ± 2 | 70 ± 5^{b} | 92 ± 3 | 86 ± 1^{b} | 100 ± 3 | 96 ± 1^{c} | 100 ± 7 | 100 ± 1^{c} |
| XD-14 | 64 ± 4^{b} | 58 ± 4^{b} | 60 ± 3^{c} | 42 ± 3^{c} | 83 ± 1^{a} | 77 ± 1^{b} | 90 ± 1 | 80 ± 5 | 91 ± 7 | 84 ± 2 |
| XD-15 | 100 ± 2 | 62 ± 3^{b} | 59 ± 3^{c} | 40 ± 3^{c} | 79 ± 2^{a} | 72 ± 3^{b} | 89 ± 6 | 70 ± 3^{b} | 81 ± 4 | 70 ± 9^{b} |
| XD-17 | 52 ± 2^{c} | 24 ± 3^{c} | 82 ± 2^{b} | 27 ± 3^{c} | 87 ± 6^{a} | 40 ± 1^{c} | 83 ± 4 | 39 ± 1^{c} | 91 ± 9 | 25 ± 2^{c} |
| XD-18 | 91 ± 4^{a} | 40 ± 4^{c} | 92 ± 1^{a} | 30 ± 2^{c} | 96 ± 3 | 55 ± 1^{c} | 100 ± 3 | 71 ± 3^{c} | 98 ± 10 | 32 ± 4^{c} |
| XD-20 | 100 ± 4 | 84 ± 4^{a} | 79 ± 2^{b} | 70 ± 7^{b} | 100 ± 5 | 100 ± 2 | 100 ± 5 | 96 ± 1 | 85 ± 2^{b} | 82 ± 1 |
| XD-22 | 100 ± 4 | 100 ± 4 | 89 ± 1^{a} | 65 ± 4^{c} | 100 ± 3 | 100 ± 8 | 100 ± 2 | 100 ± 4 | 100 ± 2 | 100 ± 1 |
| XD-23 | 81 ± 2^{a} | 48 ± 2^{c} | 75 ± 2^{b} | 72 ± 2^{b} | 100 ± 2 | 100 ± 2 | 100 ± 2 | 95 ± 3 | 100 ± 2 | 98 ± 2 |
| XD-25 | 100 ± 4 | 89 ± 4^{a} | 79 ± 2^{b} | 66 ± 5^{c} | 100 ± 5 | 90 ± 4^{a} | 100 ± 2 | 100 ± 2 | 100 ± 2 | 97 ± 2 |
| XD-26 | 86 ± 2^{a} | 56 ± 2^{c} | 100 ± 2 | 92 ± 2^{a} | 100 ± 2 | 100 ± 2 | 87 ± 2^{a} | 70 ± 2^{b} | 100 ± 2 | 92 ± 2 |
| XD-28 | 99 ± 4 | 81 ± 4^{b} | 100 ± 4 | 88 ± 4^{b} | 100 ± 4 | 96 ± 4 | 94 ± 4 | 92 ± 4 | 100 ± 4 | 98 ± 4 |
| XD-29 | 72 ± 2^{b} | 52 ± 2^{c} | 100 ± 2 | 96 ± 2 | 100 ± 2 | 98 ± 2 | 96 ± 2 | 90 ± 2^{a} | 100 ± 2 | 88 ± 2^{b} |
| XD-30 | 96 ± 2 | 77 ± 2^{b} | 100 ± 2 | 100 ± 2 | 100 ± 2 | 94 ± 2 | 81 ± 2^{b} | 79 ± 2^{b} | 100 ± 2 | 92 ± 2 |
| XD-31 | 100 ± 1 | 41 ± 1^{c} | 63 ± 1^{c} | 59 ± 1^{c} | 100 ± 1 | 90 ± 1 | 74 ± 1^{b} | 42 ± 1^{c} | 100 ± 1 | 100 ± 1 |
| XD-32 | 80 ± 7^{b} | 35 ± 7^{c} | 65 ± 7^{c} | 53 ± 7^{c} | 100 ± 7 | 74 ± 7^{b} | 73 ± 7^{b} | 30 ± 7^{c} | 100 ± 7 | 95 ± 7 |
| XD-33 | 100 ± 2 | 100 ± 2 | 97 ± 2 | 95 ± 2 | 100 ± 2 | 95 ± 2 | 97 ± 2 | 98 ± 2 | 100 ± 2 | 95 ± 2 |
| XD-34 | 100 ± 6 | 100 ± 6 | 98 ± 6 | 94 ± 6^{a} | 100 ± 6 | 88 ± 6^{a} | 98 ± 6 | 97 ± 6 | 100 ± 6 | 99 ± 6 |
| XD-35 | 100 ± 2 | 93 ± 2^{a} | 95 ± 2 | 93 ± 2^{a} | 100 ± 2 | 93 ± 2^{a} | 94 ± 2 | 93 ± 2 | 100 ± 2 | 100 ± 2 |
| XD-36 | 100 ± 3 | 92 ± 3^{a} | 93 ± 3^{a} | 91 ± 3^{a} | 100 ± 3 | 81 ± 3^{b} | 93 ± 3 | 85 ± 3^{a} | 100 ± 3 | 87 ± 3^{a} |

^{a}p < 0.05;
^{b}p < 0.01;
^{c}p < 0.001.

### Example 4. Therapeutic Effects on Liver Injury

Methods: Acetaminophen (APAP)-induced liver injury in mouse is a commonly used model to study drugs protecting liver. Overdose of APAP causes liver injury by inducing the production of reactive oxygen species and reactive nitrogen species, and excessive consumption of reductive substances such as antioxidant glutathione (GSH), leading to the reduction of GSH in vivo, and the following upregulation of the activities of the aspartate aminotransferase (AST), the alanine aminotransferase (ALT) and the lactate dehydrogenase (LDH), which will result in liver inflammation and necrosis. In this example, the APAP-induced liver injury was used to detect the protection and repair functions of our compounds in liver injury.

One-year or 8-week age mice were maintained under environmentally controlled conditions with free access to standard chow diet and water. Animal experiments were conducted in the barrier facility of the Laboratory Animal Center, Xiamen University, approved by the Institutional Animal Use and Care Committee of Xiamen University, China.

Compounds were solved with DMSO and then prepared to work concentration with 40% HBC (2-hydroxypropyl-β-cyclodextrin) in which the final work concentrations of compounds are 5 mg/kg body weight, 10 mg/kg or 20 mg/kg in 100 μl injection volume and the concentration of DMSO is 10%. Compounds were intraperitoneal (i.p.) injected once daily for five days. For Feroline and Tschimganine, one-year age mice were used in the experiment. For other compounds, 8-week age mice were used. For Feroline, Tschimgine, Tschimganidine and hedragonic acid, 10 mg/kg dose of compounds were administered to mice. For Tschimganine, 20 mg/kg dose of compound was administered to mice. For other compounds, 5 mg/kg dose of compounds were administered to mice. Six hours after the fifth injection, 500 mg/kg body weight of APAP solved in PBS was i.p. injected to the mice. 24 hours later, mice were sacrificed. Part of each liver was fixed in 4% paraformaldehyde, and the liver histology characterization was analyzed by haematoxylin and eosin (H&E) staining with paraffin-embedded sections by standard procedures. Other liver tissues were collected for detecting the GSH levels, and the mRNA expression of genes involved in liver repairing, such as GPX1 and UGT1a1, by RT-PCR. The serums were collected to measure enzymes activities including AST, ALT and LDH. For the synthetic derivatives, ALT and GSH were selected as indicating markers for the function of these compounds.

Results:

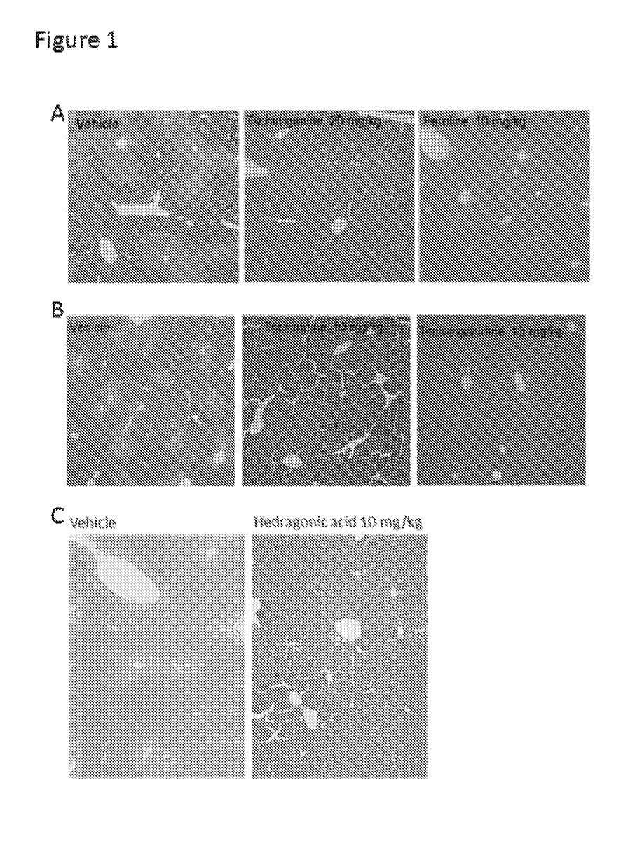

As in FIG. 1, the pathological sections in control group displayed obvious cell infiltration, vacuolization and necrosis in hepatic lobule. There were a large number of inflammatory cell infiltration, cell turbidity, dissolved karyopycnosis or broken in lobules and portal area. And liver cell cords were also blurred. Compared to the severely liver injury in the control group, the livers in mice treated with Feroline, Tschimganine, Tschimgine, Tschimganidine and hedragonic acid displayed almost normal liver morphology. The activities of serum AST, ALT and LDH were dramatically lower (Table 5), the GSH levels in liver tissues were increased (Table 6), and the expression levels of GPX1 and UGT1α1, genes involved in liver repairing, were significantly upregulated than that in the vehicle control group (Table 5), which further indicated the protection and repair functions of the compounds Feroline, Tschimganine, Tschimgine, Tschimganidine and hedragonic acid in APAP-induced liver injury. The serum ALT activities in mice treated with the synthetic derivatives or analogues were also significantly decreased (Table 7), and the GSH levels were increased (Table 8), both indicating that the synthetic derivatives also have these functions. This example demonstrated that our compounds have therapeutic effects on liver injury.

TABLE 6
Compounds treatment can increase the hepatic
GSH levels of mice hurt by APAP.

| | GSH |
|---|---|
| Compounds | (μM/g liver tissue) |
| Vehicle Control | 8.0 ± 2.8 |
| Feroline (10 mg/kg) | 14.2 ± 1.6^{b} |
| Tschimganine (20 mg/kg) | 14.9 ± 0.6^{b} |

^{b}p < 0.01.

TABLE 7
Synthetic derivatives can protect mice from
APAP-induced liver injury.

| Compound treatment | ALT |
|---|---|
| (5 mg/kg) | (U/L) |
| Vehicle Control | 786 ± 111 |
| XD-4 | 236 ± 65 ^{a} |
| XD-5 | 197 ± 57 ^{b} |
| XD-6 | 109 ± 26 ^{c} |
| XD-8 | 115 ± 38 ^{c} |
| XD-10 | 114 ± 31 ^{c} |
| XD-16 | 127 ± 46 ^{b} |
| XD-17 | 117 ± 39 ^{c} |
| XD-18 | 135 ± 48 ^{a} |
| XD-23 | 130 ± 62 ^{a} |
| XD-28 | 114 ± 25 ^{c} |
| XD-29 | 118 ± 31 ^{c} |
| XD-30 | 107 ± 41 ^{b} |

^{a} p < 0.05; ^{b} p < 0.01; ^{c} p < 0.001.

TABLE 8
Synthetic derivatives treatment can increase the hepatic
GSH levels of mice hurt by APAP.

| Compounds | GSH |
|---|---|
| (5 mg/kg) | (μM/g liver tissue) |
| Vehicle Control | 9.1 ± 0.5 |
| XD-0 | 15.0 ± 0.5 ^{b} |
| XD-5 | 12.8 ± 0.3 ^{b} |
| XD-11 | 12.6 ± 0.4 ^{b} |
| XD-12 | 12.5 ± 0.7 ^{a} |
| XD-13 | 13.7 ± 0.9 ^{a} |
| XD-35 | 12.6 ± 1.6 ^{a} |
| XD-36 | 14.8 ± 0.3 ^{b} |

^{a} p < 0.05, ^{b} p < 0.01.

### Example 5. Therapeutic Effects on Metabolic Diseases

Methods:

KK-Ay mice (KK/Upj-Ay/J) are animal models with moderate obese and diabetes with insulin resistance. KK-Ay mice develop hyperglycemia, hyperinsulinemia, glucose intolerance and obesity as well as fat accumulation in liver by eight weeks of age. Pancreatic islets are hypertrophied and the β-cells are degranulated. db/db mice (BKS.Cg-Dockr+/+ Lepr^{db}/Jnju) are animal models of type II diabetes. They have the phenotype of insulin resistance. These model mice were used to detect the functions of our compounds on metabolic diseases.

8-10 weeks age mice were maintained as example 4, and fed with high-fat diet (Research Diets, D12492). The doses of the compounds used are 10 mg/kg, 20 mg/kg or 50 mg/kg body weight indicated in Table 9 to 14. Mice were i.p. injected with compounds once daily for 7, 10, 11 or 14 days as indicated in each table. After the last compounds injection, mice were fasted for 6 hours with free access to water, and then sacrificed. Part of each liver was fixed in 4% paraformaldehyde for H&E staining, and other liver tissues were stored in liquid nitrogen for enzyme activity measurement and gene expression analysis by RT-PCR. The serums were collected to measure metabolic parameters, including serum glucose, insulin, cholesterol, free fatty acid (FFA) and triglyceride levels. Serum glucose was analyzed using glucose oxidase method (Applygen, Beijing, China). The blood glucose in Table 11 was measured with Berenger blood glucose test strips (B/BRAUN, German) from blood by cutting mice tail after the 6^{th} injection and following fast for 16 hours. Serum cholesterol and FFA were analyzed using Cholesterol Assay Kit and FFA Assay Kit (Bioassay Systems, USA; Nanjing Jiancheng Bioengineering Institute, China; FFA ELISA, R&D, USA), respectively. Serum triglyceride was analyzed using Triglyceride Assay Kit (Bioassay Systems, USA; WAKO Chemicals Inc., Japan; Applygen, Beijing, China). Liver triglyceride was analyzed using Tissue triglyceride assay kit (Applygen, Beijing, China). Serum insulin levels were measured by the Ultra Sensitive Mouse Insulin ELISA Kit (Crystal Chem. Inc., USA). RNA was isolated using Tissue RNA kit (Omega Bio-Tek, GA). The first strand cDNA were obtained by TAKARA reverse transcription kit. Real-time quantitative PCR were performed on a CFX96™ Real-Time PCR Detection System (Bio-Rad) using SYBR Premix Ex Taq™ (TAKARA). Relative mRNA expression levels were normalized to actin levels.

Results:

As shown in Table 9 to 13, the serum/blood glucose levels, insulin levels, cholesterol levels, FFA levels and/or the triglyceride levels were significantly lowered in FXR-ligand compounds treated mice, consistent with the genes regulation related to glucose and lipid metabolism, indicating the therapeutic effects of our compounds in metabolic diseases, including diabetes, obesity, hyperglycemia, hypertriglyceridemia, hypercholesterolemia, hyperinsulinemia, insulin resistance, etc. High levels of blood triglycerides and glucose are the alert indicators of cardiovascular disease. These indicators reflect the high risk for development of cardiovascular disease. In this example, the FXR-ligand compounds treatment significantly decreased the blood levels of glucose and triglyceride, indicating their therapeutic effects in cardiovascular diseases. Studies demonstrated that high total cholesterol level is positively correlated with the degree of carotid atherosclerotic plaque lesions. In this example, some compounds can efficaciously decrease the serum cholesterol levels, indicating their therapeutic effects on atherosclerosis.

TABLE 9
Feroline and tschimganine treatment down-regulated the metabolic parameters in serum
and mRNA levels of genes related to metabolism in liver of diabetic KK-Ay mice.

| Compound treatment | | Feroline | | Tschimganine |
|---|---|---|---|---|
| (for 14 days) | Vehicle | (20 mg/kg) | Vehicle | (20 mg/kg) |
| Glucose (mg/dl) | 253 ± 22 | 177 ± 16^{a} | 234 ± 25 | 123 ± 10^{b} |
| Cholesterol (mg/dl) | 140 ± 14 | 116 ± 10^{a} | / | / |
| Triglyceride (mg/dl) | 170 ± 20 | 136 ± 8^{a} | 170 ± 18 | 134 ± 15^{a} |
| FFA (μM) | 218 ± 25 | 122 ± 30^{b} | 210 ± 24 | 151 ± 14^{a} |

mRNA levels in liver

| | | | | |
|---|---|---|---|---|
| G6PC | 1 ± 0.1 | 0.66 ± 0.07^{a} | 1 ± 0.08 | 0.59 ± 0.08^{a} |
| GK | 1 ± 0.1 | 0.74 ± 0.11 | 1 ± 0.04 | 0.54 ± 0.07^{a} |
| SREBP1c | 1 ± 0.15 | 0.3 ± 0.08^{b} | 1 ± 0.07 | 0.38 ± 0.05^{a} |
| CHREBP | 1 ± 0.07 | 0.4 ± 0.1^{b} | 1 ± 0.11 | 0.43 ± 0.08^{a} |
| FDFT1 | 1 ± 0.04 | 0.75 ± 0.1^{a} | 1 ± 0.06 | 0.77 ± 0.06^{a} |

^{a}p < 0.05; ^{b}p < 0.01.
/ not test.

TABLE 10
Hedragonic acid treatment downregulated the serum
glucose levels of diabetic KK-Ay mice.

| Compound treatment | | Hedragonic acid |
|---|---|---|
| for 14 days | Vehicle | (10 mg/kg) |
| Serum Glucose (mg/dl) | 253 ± 11 | 161 ± 21^{b} |

^{b}p < 0.01.

TABLE 11
Tschimgine treatment downregulated the metabolic
parameters in serum of db/db mice.

| Compound treatment for | | Tschimgine |
|---|---|---|
| 11 days | Vehicle | (10 mg/kg) |
| Blood Glucose (mg/dl) | 452 ± 24 | 252 ± 54^{b} |
| Serum Cholesterol (mg/dl) | 121 ± 13 | 96 ± 3^{a} |
| Serum FFA (μM) | 371 ± 27 | 302 ± 21^{a} |
| Serum Triglyceride | 96 ± 16 | 38 ± 8^{a} |
| (mg/dl) | | |

^{a}p < 0.05; ^{b}p < 0.01.

TABLE 12
Tschimganine and hedragonic acid down-regulated serum levels
of glucose and insulin in db/db mice.

| Compound treatment | | Tschimganine | Hedragonic acid |
|---|---|---|---|
| for 7 days | Vehicle | (10 mg/kg) | (50 mg/kg) |
| Serum Insulin (ng/ml) | 12 ± 3 | 4.7 ± 0.4^{b} | 4.65 ± 0.2^{b} |
| Serum Glucose (mg/dl) | 521 ± 54 | 342 ± 36^{a} | 334 ± 50^{a} |

^{a}p < 0.05; ^{b}p < 0.01.

TABLE 13
Compounds reduce the serum glucose
levels in db/db mice.

| Compounds | |
|---|---|
| (10 mg/kg) | Serum Glucose |
| (treat for 10 days) | (mg/dl) |
| Vehicle | 527 ± 63 |
| XD-0 | 223 ± 33^{b} |
| XD-9 | 399 ± 41^{a} |
| XD16 | 404 ± 46^{a} |
| XD20 | 396 ± 55^{a} |
| XD24 | 307 ± 39^{a} |
| XD25 | 389 ± 47^{a} |
| XD30 | 400 ± 51^{a} |
| XD31 | 326 ± 51^{a} |
| XD33 | 400 ± 23^{a} |
| XD35 | 337 ± 67^{a} |
| XD36 | 366 ± 65^{a} |
| Ferutinin | 258 ± 56^{b} |

^{a}p < 0.05, ^{b}p < 0.01.

Kidney is the main organ for urea excretion. After urine is filtrated in glomerular, urea can be reabsorbed in renal tubules. The faster urine flow in the renal tubules, the less urea is reabsorbed. If the kidney is injured, the filtration ratio of urine in glomerular will decrease. The blood urea nitrogen (BUN) concentration will increase rapidly when the filtration ratio in glomerular decrease lower than 50%. Various renal parenchymal diseases, including glomerulonephritis, interstitial nephritis, acute and chronic renal failure, renal lesions and renal destructive lesions, can increase the BUN levels. Therefore, BUN is a main indicator for kidney function, as well as the uremia.

Chronic kidney diseases are included in the complications of diabetes. The BUN levels were measured with the Urea Assay kit (Nanjing Jiancheng Bioengineering Institute, China) in this example, and the results showed that the FXR-ligand compounds significantly decreased the BUN levels in diabetic db/db mice (Table 14), demonstrating the therapeutic effects of our compounds in various kidney diseases including glomerulonephritis, interstitial nephritis, acute and chronic renal failure, renal lesions, renal destructive lesions and uremia that with increased BUN.

### Example 6. Therapeutic Effects on NAFLD and Cirrhosis

Non-alcoholic fatty liver disease (NAFLD), caused by accumulation of abnormal amounts of fat in the liver, not due to excessive alcohol consumption, has emerged as a serious metabolic disorder. Patients with NAFLD have a variety of hepatic dysregulation ranging from abnormal triglyceride accumulation in hepatocytes (steatosis) to steatohepatitis (non-alcoholic steatohepatitis, NASH) with fibrosis, which may evolve to cirrhosis and/or hepatocellular carcinoma. Hepatic steatosis is present in up to one-third of adults in developed countries including an increasing prevalence in young people, directly contributing to liver disease. For example, NAFLD-induced liver failure is a leading indication for liver transplantation. Moreover, NAFLD has become a meaningful predictive factor of death from cardiovascular diseases, as well as of the onset of type 2 diabetes and chronic kidney disease. These considerations strongly indicate the necessity to treat each stage of NAFLD.

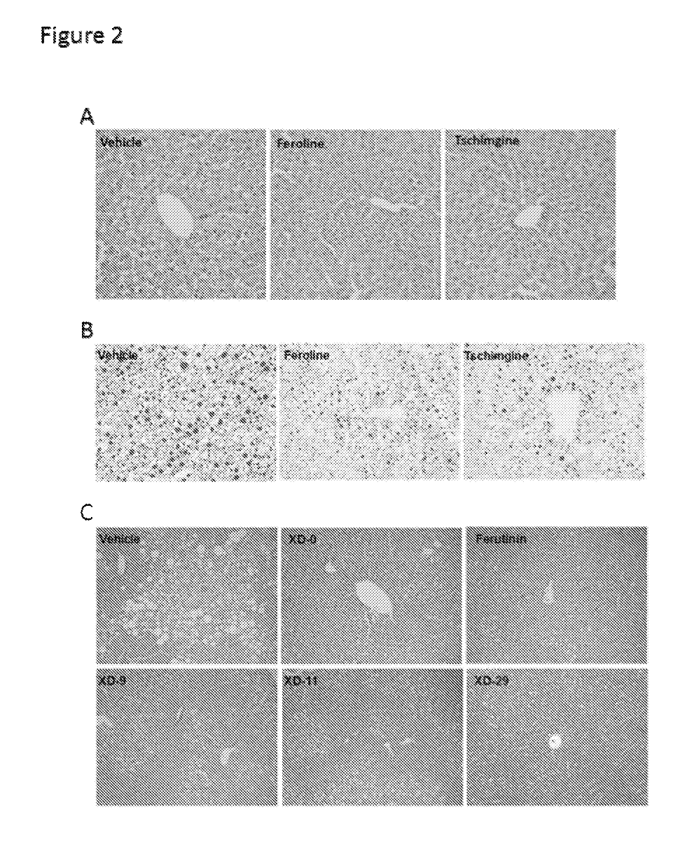

Liver tissues fixed in 4% paraformaldehyde in example 5 were performed for H&E staining by standard procedures. Liver tissues fixed in 4% paraformaldehyde were embedded in optimum cutting temperature compound (OCT), and cryosectioned. Frozen liver sections were stained with 0.3% oil red O according to standard procedures. Histological examination of liver sections by H&E staining showed the extensive existence of vesicular hepatocyte vacuolation in vehicle treated control mice (FIG. 2). However, FXR-ligand compounds treatment nearly completely reversed the hepatic steatosis in the diabetic mice, the tissue lipid accumulation disappeared, and the liver cells showed tight compact structure (FIG. 2).

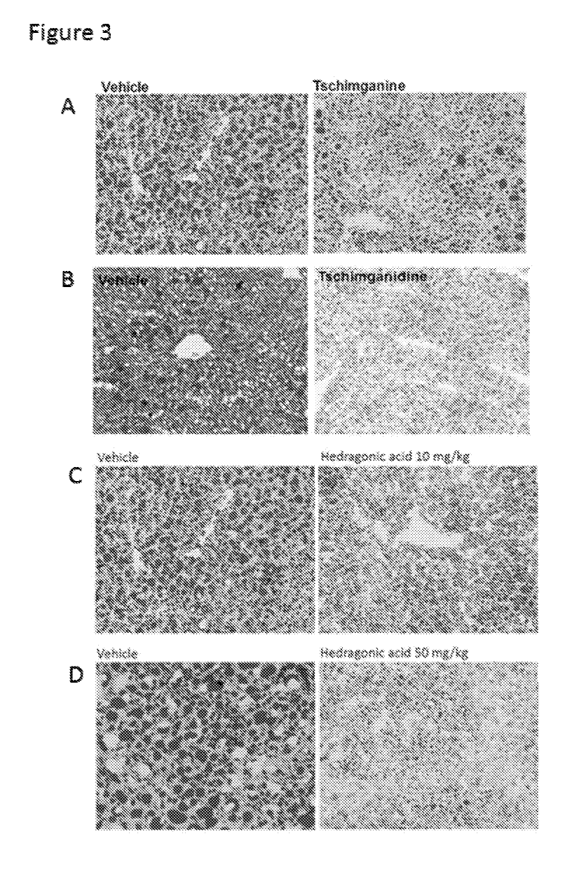

Oil Red O is a fat-soluble dye, and it is highly soluble in fat, which can make the triglyceride and other neutral fat coloring in red. The Oil Red O staining is commonly used in pathological diagnosis to show the fat in tissue. To analyze the status of fat accumulation in mice livers, Oil Red O staining was performed in the liver sections from mice treated in example 5 (FIGS. 2 and 3). Liver sections from vehicle treated mice showed abundant lipid accumulation, especially containing many large lipid droplets. While liver sections from mice treated with FXR-ligand compounds dramatically reduced the lipid accumulation, where the large lipid droplets nearly disappeared.

The triglyceride levels in liver tissues from mice treated with compounds were analyzed using Tissue triglyceride assay kit (Applygen, Beijing, China). The results in Table 15 illustrate the hepatic triglyceride levels were reduced in high-fat diet fed db/db mice treated with FXR-ligand compounds. The serum ALT activities further indicated the safety and hepatoprotection functions of these compounds for mice (Tables 15 & 17).

TABLE 15
The hepatic triglyceride levels and serum ALT
activities were reduced in high-fat diet fed db/db
mice treated with compounds disclosed.

| Compounds | Hepatic | |
|---|---|---|
| (10 mg/kg | triglyceride | Serum ALT |
| for 10 days) | (mM/g) | (U/L) |
| Vehicle Control | 148 ± 14 | 377 ± 48 |
| XD-0 | 91 ± 22 ^{a} | 106 ± 22 ^{a} |
| XD-3 | 80 ± 19 ^{a} | 185 ± 29 ^{a} |
| XD-6 | 90 ± 11^{b} | 101 ± 28 ^{a} |
| XD-8 | 102 ± 8 ^{c} | 115 ± 31 ^{a} |
| XD-9 | 93 ± 14 ^{a} | 121 ± 33 ^{a} |
| XD-11 | 102 ± 13 ^{a} | 106 ± 26 ^{a} |
| XD-12 | 103 ± 20 ^{a} | 199 ± 37 ^{a} |
| XD-16 | 81 ± 23 ^{a} | 104 ± 33 ^{a} |
| XD-20 | 60 ± 15^{b} | 115 ± 29 ^{a} |
| XD-25 | 116 ± 5 ^{c} | 124 ± 21 ^{a} |
| XD-28 | 87 ± 10 ^{c} | 103 ± 17^{b} |
| XD-29 | 116 ± 11 ^{a} | 101 ± 46^{b} |
| XD-30 | 108 ± 8 ^{c} | 92 ± 27 ^{a} |
| Ferutinin | 67 ± 15^{b} | 87 ± 15^{b} |

^{a} p < 0.05; ^{b}p < 0.01; ^{c} p < 0.001.

Triglyceride accumulation is due to the imbalance between triglyceride synthesis and clearance. One key gene controlling hepatic lipogenesis is SREBP-1c, whose up-regulation has been implicated in occurrence of hepatic steatosis. Liver-specific inhibition of ChREBP improves hepatic steatosis and insulin resistance in ob/ob mice (see e.g., Renaud (2006) Diabetes, 55(8):2159-70.). Quantitative PCR data (Table 9) revealed that FXR-ligand compounds like feroline and tschimganine treatment decreased the hepatic mRNA levels of SREBP-1c and ChREBP. The gene expression pattern in KK-Ay mice liver further explained the underlying molecular mechanism for the reduction of lipid accumulation by the FXR-ligand compounds (Table 9).

The data demonstrated that our FXR-ligand compounds effectively reduced the lipid accumulation in mice liver. Hypertriglyceridemia and hypercholesterolemia are closely related with liver steatosis and atherosclerosis. The data of serum triglyceride and cholesterol levels of mice here support the functions of our compounds in treating NAFLD.

NAFLD with excessive fat accumulation in liver will affect the blood and oxygen supplies to liver and the metabolism of liver organ, resulting in amounts of cell swelling, inflammatory infiltration and necrosis in liver. Once fibrosis and false lobules appear, cirrhosis will happen and the risk of liver cancer will be greatly increased. The levels of various collagen contents are higher in patients with liver cirrhosis. Masson's staining is a three-color staining protocol used in histology. It is widely used to study hepatic pathologies (cirrhosis). It's an authoritative and classic method to detect the existence and extend of accumulation of the collagen fiber. Sirius Red staining is also presented as a method for collagen determination. In this example, the Masson's staining and Sims red staining were performed to detect the collagen deposit in high fat diet fed db/db mice in Example 5. The Masson's staining kit (Nanjing Jiancheng Engineering Institute, China) was used for the staining of liver sections in Example 5. The Sirius red staining is performed as standard procedure. RT-PCR was used detect the expression of collagen and related genes such as α1(I) collagen, α2(I)collagen, α-SMA and MCP-1 in mice livers.

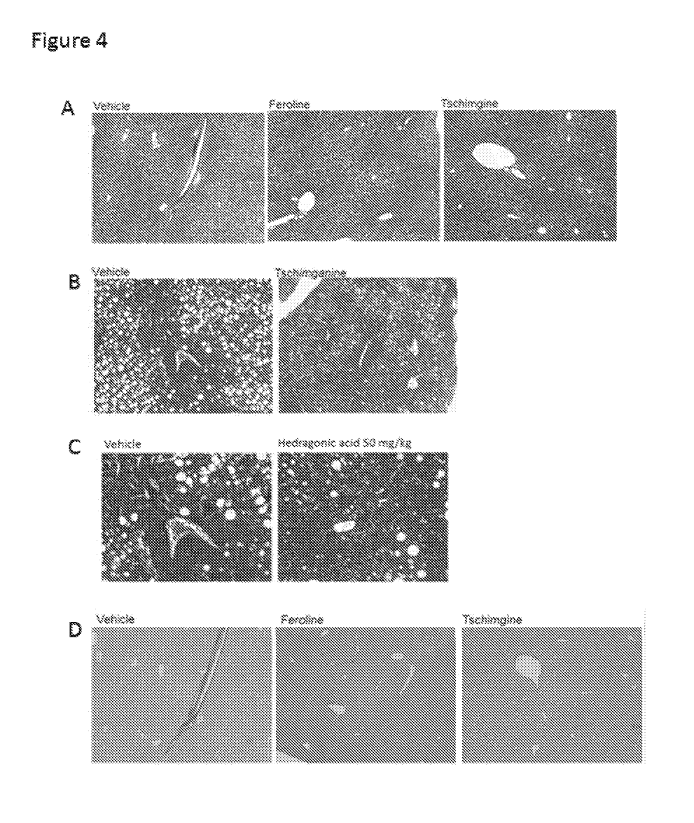

As shown in FIG. 4, liver sections from compounds treated mice showed no obvious collagen deposit compared to the existing blue collagen deposit around the blood vessel in the vehicle control mice liver sections. In FIG. 4D, compounds treated mice liver also showed no obvious collagen colored in red compared to the vehicle control samples. Correspondingly, the expression levels of collagen and related genes such as α1(I) collagen, α2(I) collagen, α-SMA and MCP-1 were significantly decreased in compounds treated mice livers (Table 16).

Taken together, the data from H&E staining, oil red O staining, serum and tissue triglyceride assay kit, Masson's staining, Sirius red staining and gene expression assay demonstrated that our compounds have therapeutic effects on NAFLD, NASH and cirrhosis, and can be used to prevent NAFLD, NASH, cirrhosis and even liver cancer.

TABLE 16
Relative mRNA levels of collagen related genes in
mice treated in Example 5.

| Compound | α1(I) | α2(I) | | |
|---|---|---|---|---|
| treatment | collagen | collagen | α-SMA | MCP-1 |
| Vehicle | 1.1 ± 0.2 | 1.1 ± 0.1 | 1 ± 0.1 | 1.2 ± 0.2 |
| Feroline | 0.37 ± 0.1^{a} | 0.6 ± 0.06^{a} | 0.73 ± 0.06^{a} | / |
| (10 mg/kg) | | | | |
| Tschmganine | 0.6 ± 0.12^{a} | / | 0.56 ± 0.12^{a} | 0.62 ± 0.13^{a} |
| (10 mg/kg) | | | | |
| Tschimgine | 0.6 ± 0.13^{a} | 0.75 ± 0.9^{a} | 0.47 ± 0.1^{a} | / |
| (10 mg/kg) | | | | |
| Hedragonic acid | 0.75 ± 0.1^{a} | 0.84 ± 0.1 | 0.5 ± 0.1^{a} | 0.7 ± 0.1^{a} |
| (50 mg/kg) | | | | |

^{a}p < 0.05, / not test.

### Example 7. Therapeutic Effects on Cholestasis

Alkaline Phosphatase (ALP) is a marker of cholestasis. Cholestasis can be suspected when there is an elevation of ALP enzymes. In fact, greater than 90% of patients with bile stasis will have an elevated alkaline phosphatase. ALT is found predominantly in the cytosol of hepatocytes, and an elevated ALT is more likely to suggest liver injury. The aminotransferase is used to evaluate the presence of hepatitis and may be elevated in cholestasis or with common bile duct obstruction. In this example, 8-10 week age db/db mice were fed with high-fat diet and treated with 10 mg/kg of compounds once daily for 10 days as in Example 5. The activities of serum ALP and ALT were analyzed using ALP kit and ALT kit, respectively (Nanjing Jiancheng Engineering Institute, China). As indicated in Table 17, compounds treatment efficaciously decreased the activities of serum ALP and ALT, demonstrating the therapeutic effects of compounds on cholestasis.

TABLE 17
The activities of serum ALP and ALT in
db/db mice treated with compounds.

| | | ALP |
|---|---|---|
| Compounds | Serum | Serum ALT |
| (10 mg/kg) | (U/L) | (U/L) |
| Vehicle Control | 129 ± 15 | 377 ± 48 |
| XD-13 | 92 ± 7^{c} | 135 ± 5^{c} |
| XD-14 | 86 ± 20^{a} | 62 ± 11^{c} |
| XD-16 | 88 ± 10^{b} | 104 ± 33^{a} |
| XD-17 | 76 ± 17^{a} | 185 ± 51^{a} |
| XD-18 | 77 ± 9^{c} | 129 ± 19^{b} |
| XD-20 | 73 ± 19^{a} | 115 ± 29^{a} |
| XD-29 | 69 ± 9^{c} | 101 ± 46^{b} |
| XD-30 | 64 ± 14^{a} | 92 ± 27^{a} |
| XD-32 | 83 ± 23^{a} | 110 ± 42^{a} |
| XD-34 | 91 ± 18^{a} | 73 ± 20^{b} |

^{a}p < 0.05; ^{b}p < 0.01; ^{c}p < 0.001.

### Example 8. Therapeutic Effects on Inflammatory Disease

Inflammatory cytokines are produced during inflammation and substance secreted by the cells involved in the inflammatory response. Inflammatory cytokines are markers of the inflammatory reaction. For example, the expression of iNOS (inducible nitric oxide synthase) is one of the direct consequences of an inflammatory process. Studies performed in rodents mostly imply that iNOS activity plays a detrimental role in chronically inflammatory processes. (see e.g., Kroncke et al., (1998) Clin Exp Immunol. 113(2): 147-156.)

In this example, we detected the levels of various inflammatory cytokines including iNOS, IFNγ, TGFβ, TNFα, COX2, IL-1β, IL-6, SAA1, MIP-1α and CD36 in liver, kidney, white adipose tissue (WAT) in various mice models, including wild type mice treated with APAP in example 4, high-fat diet fed diabetic and obesity db/db and KK-Ay mice in example 5, high-fat diet fed wild-type mice in Example 5, as well as in primary hepatocytes with LPS induced inflammation, using RT-PCR.

As shown in Table 18 to Table 22, compounds treatment significantly decreased the inflammatory cytokines levels in liver of mice with diabetes and liver injury induced by APAP, demonstrating the therapeutic effects of compounds on inflammatory diseases caused by chemical drugs.

As shown in Table 18, Table 19, Table 23 and Table 24, compounds treatment significantly decreased the inflammatory cytokines levels in liver tissues, kidney tissues, colon tissues and/or WAT in high-fat diet fed diabetic and obesity db/db and KK-Ay mice and high-fat diet fed wild-type mice, demonstrating the therapeutic effects of compounds on inflammatory diseases complicated with diabetes and obesity.

Lipopolysaccharides (LPS), also known as lipoglycans and endotoxins in the outer membrane of Gram-negative bacteria, elicit strong immune responses in animals. In this example, primary hepatocytes were extracted from wild type C57B6/J mice, and were treated with 20 μM of compounds for 18 hours following 20 μg/ml of LPS treatment for 6 hours. The expression of inflammatory cytokines was detected using RT-PCR. As shown in Table 25, compounds treatment significantly decreased the inflammatory cytokines levels in primary hepatocytes with LPS-induced inflammation, demonstrating the therapeutic effects of compounds on inflammatory diseases caused by bacteria (LPS).

TABLE 23
Compounds treatment decreased the inflammatory cytokines
levels in tissues of high-fat diet fed db/db mice.

| | mRNA level of iNOS (fold) | |
|---|---|---|
| Compounds (5 mg/kg) | Colon tissue | Liver tissue |
| Vehicle Control | 1.12 ± 0.23 | 1.06 ± 0.14 |
| XD-4 | 0.53 ± 0.37^{a} | 0.41 ± 0.1^{a} |
| XD-10 | 0.49 ± 0.21^{a} | 0.52 ± 0.3^{a} |
| XD-15 | 0.41 ± 0.16^{a} | 0.47 ± 0.21^{a} |
| XD-16 | 0.42 ± 0.22^{a} | 0.39 ± 0.18^{a} |
| XD-20 | 0.5 ± 0.18^{a} | 0.51 ± 0.2^{a} |
| XD-23 | 0.55 ± 0.29^{a} | 0.2 ± 0.1^{a} |
| XD-32 | 0.45 ± 0.13^{a} | 0.22 ± 0.12^{a} |
| Ferutinin | 0.35 ± 0.16^{a} | 0.44 ± 0.2^{a} |

^{a}p < 0.05.

TABLE 24
Compounds treatment decreased the inflammatory cytokines
levels in WAT of high-fat diet fed db/db mice.

| | WAT, mRNA level (fold) | |
|---|---|---|
| Compound (5 mg/kg) | TNFα | IL-1β |
| Vehicle Control | 1.15 ± 0.13 | 1.06 ± 0.2 |
| XD-5 | 0.56 ± 0.17^{a} | 0.33 ± 0.2^{a} |
| XD-23 | 0.6 ± 0.21^{a} | 0.64 ± 0.13^{a} |
| Ferutinin | 0.69 ± 0.11^{a} | 0.37 ± 0.15^{a} |

^{a}p < 0.05.

### Example 9. Anti-Oxidation Effects

The reduced glutathione (GSH) is a natural antioxidant in cells and plays important function responding the oxidative stress. The GSH levels will decrease with age, infection, poisoning, exogenous toxins and oxidative stress. The Sestrin2 (Sesn2) gene encodes a conserved antioxidant protein that is induced on oxidative stress and protects cells against reactive oxygen species. SOD2 is an antioxidant enzyme. The antioxidants are defense system for organ to prevent damage by free radical. In this example, the mouse embryonic fibroblasts (MEFs) were treated with compounds for 24 hours, and the GSH levels were analyzed using GSH kit (Nanjing Jiancheng Engineering Institute, China). The results showed significantly increased GSH levels in cells treated with compounds compared to the control (Table 26 and Table 27). Correspondingly, the expression levels of the antioxidant genes such as SOD2 and SESN2 increased in compounds treated MEFs (Table 26 and Table 28).

TABLE 27
Hedragonic acid treatment increased the
GSH levels in MEFs.

| Compounds | GSH (μM) |
|---|---|
| Vehicle Control | 31 ± 6 |
| Hedragonic acid | 72 ± 9^{a} |
| (20 μM) | |

^{a}p < 0.05.

TABLE 28
Compounds treatment increased the mRNA levels of
antioxidant gene SOD in livers of db/db mice.

| Compounds | mRNA level of |
|---|---|
| (10 mg/kg) | SOD2 (fold) |
| Vehicle Control | 1 ± 0.3 |
| XD-4 | 1.6 ± 0.1 ^{b} |
| XD-16 | 1.5 ± 0.1 ^{b} |
| XD-17 | 1.8 ± 0.3 ^{a} |

^{a} p < 0.05, ^{b} p < 0.01.

Free radicals are atoms or groups of atoms with an odd (unpaired) number of electrons and can be formed when oxygen interacts with certain molecules. Once formed, these highly reactive radicals can start a chain reaction, like dominoes. Their chief danger comes from the damage when they react with important cellular components such as DNA, or the cell membrane. Cells may function poorly or die if this occurs. Free radicals can damage the body's immune system, induce cancer, and interfere with cell repair, cell metabolism and other interference. Excessive accumulation of free radicals will lead to severely consequences such as aging, cancer, inflammation and autoimmune diseases. Hedragonic acid was selected to test the ability to clear the superoxide anion radical in vitro using method as described in patent CN 102813683 B. The result showed the clearance rate of hedragonic acid to superoxide anion radical is 58%±6.4% with p<0.01, demonstrating the FXR-ligand compound has effective ability to clear the free radical. Together with the increased GSH levels in liver from mice treated with our compounds in example 4 (Table 6 and Table 8), the data demonstrated the anti-oxidation effects of our compounds, indicating the functions of these compounds in clearing free radicals, increasing levels of reducing substances, resulting in anti-oxidation and anti-aging.

1. A method for treating an FXR-mediated process or disease in a mammal, comprising administering to the mammal a therapeutically effective amount of at least one compound having the formula: 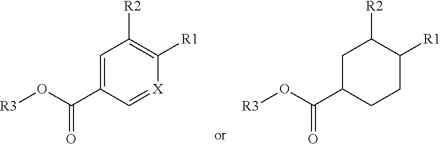
or in a pharmaceutically acceptable carrier and/or diluent form thereof, wherein R1 is independently selected from halogen, hydroxy, amino, lower amines, or methyoxy group; R2 is independently selected from hydrogen, halogen, sulfur, lower amines, methanethiolate, lower alkyl or alkoxy group; X is C, N or O; and R3 is bicyclo[2.2.1] heptane, bicyclo[2.2.1]heptane, naphthalene-decahydro, [1,7,7]-trimethyl, tricyclo[3.3.1.^{13, 7}]decane, tricyclo[3.3.1.^{13,7}]decane-1-methyl, bicyclo, trimethylbicyclo, cyclooctyl, cyclododecyl, cycloheptyl, decahydronapht, cycloalkyl, heterocyclyl, cycloalkylalkyl, or heterocyclylalkyl group.

2. The method of claim 1, wherein said compound has the formula: 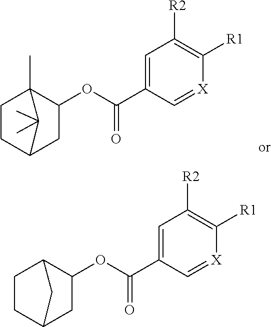
or in a pharmaceutically acceptable carrier and/or diluent form thereof,
wherein R1 is independently selected from halogen, hydroxy, amino, lower amines, or methyoxy group; R2 is independently selected from hydrogen, halogen, sulfur, lower amines, methanethiolate, lower alkyl or alkoxy group; X is C, N or O.

3. The method of claim 1, wherein said compound has the formula: 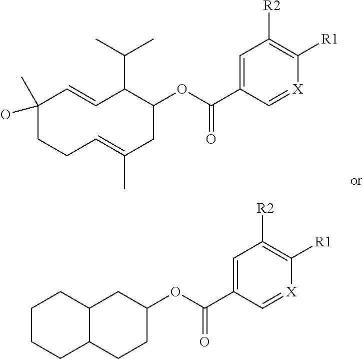
or in a pharmaceutically acceptable carrier and/or diluent form thereof,
wherein R1 is independently selected from halogen, hydroxy, amino, lower amines, or methyoxy group; R2 is independently selected from hydrogen, halogen, sulfur, lower amines, methanethiolate, lower alkyl or alkoxy group; X is C, N or O.

4. The method of claim 1, wherein said compound has the formula: 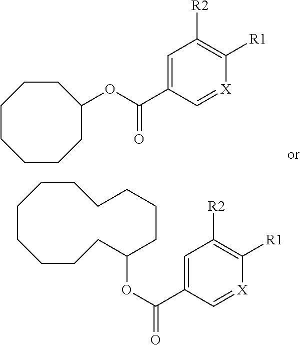
or in a pharmaceutically acceptable carrier and/or diluent form thereof,
wherein R1 is independently selected from halogen, hydroxy, amino, lower amines, or methyoxy group; R2 is independently selected from hydrogen, halogen, sulfur, lower amines, methanethiolate, lower alkyl or alkoxy group; X is C, N or O.

5. The method of claim 1, wherein said compound has the formula: 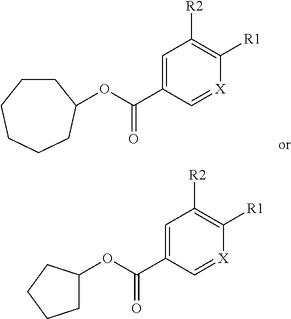
or in a pharmaceutically acceptable carrier and/or diluent form thereof,
wherein R1 is independently selected from halogen, hydroxy, amino, lower amines, or methyoxy group; R2 is independently selected from hydrogen, halogen, sulfur, lower amines, methanethiolate, lower alkyl or alkoxy group; X is C, N or O.

6. (canceled)

7. The method of claim 1, wherein said compound has the formula: 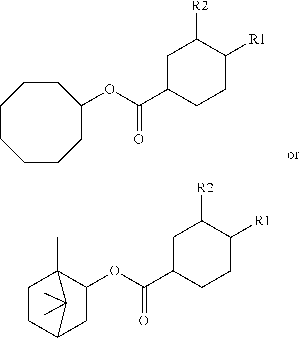
or in a pharmaceutically acceptable carrier and/or diluent form thereof,
wherein R1 is independently selected from halogen, hydroxy, amino, lower amines, or methyoxy group; R2 is independently selected from hydrogen, halogen, sulfur, lower amines, methanethiolate, lower alkyl or alkoxy group; X is C, N or O.

8. (canceled)

9. The compound according to claim 1 selected from the group consisting of the following:
Juniferdin Derivative,9;
1,7,7-trimethylbicyclo[2.2.1]heptan-2-yl 4-fluorobenzoate;
1,7,7-trimethylbicyclo[2.2.1]heptan-2-yl 4-fluoro-3-methyl benzoate;
1,7,7-trimethylbicyclo[2.2.1]heptan-2-yl 4-fluoro-3-methoxybenzoate;
1,7,7-trimethylbicyclo[2.2.1]heptan-2-yl 4-amino-3-methyl benzoate;
1,7,7-trimethylbicyclo[2.2.1]heptan-2-yl 4-aminobenzoate;
1,7,7-trimethylbicyclo[2.2.1]heptan-2-yl isonicotinate;
1,7,7-trimethylbicyclo[2.2.1]heptan-2-yl 6-oxo-1,6-dihydropyridine-3-carboxylate;
1,7,7-trimethylbicyclo[2.2.1]heptan-2-yl 6-oxo-6H-pyran-3-carboxylate;
cyclooctyl 4-hydroxybenzoate;
cyclododecyl 4-hydroxybenzoate;
cycloheptyl 4-aminobenzoate;
1,7,7-trimethylbicyclo[2.2.1]heptan-2-yl 3-bromo-4-fluorobenzoate;
1,7,7-trimethylbicyclo[2.2.1]heptan-2-yl 4-nitrobenzoate;
1,7,7-trimethylbicyclo[2.2.1]heptan-2-yl 3-methyl-4-nitrobenzoate;
cyclooctyl 4-aminobenzoate;
cyclooctyl 4-hydroxy-3-methyl benzoate;
cyclooctyl 4-hydroxy-3-methoxybenzoate;
cyclooctyl 4-hydroxycyclohexanecarboxylate;
1,7,7-trimethylbicyclo[2.2.1]heptan-2-yl 4-hydroxycyclohexanecarboxylate;
1,7,7-trimethylbicyclo[2.2.1]heptan-2-yl 4-aminocyclohexanecarboxylate;
bicyclo[2.2.1]heptan-2-yl 4-aminobenzoate;
decahydronaphthalen-2-yl 4-aminobenzoate;
cyclooctyl 4-nitrobenzoate;
bicyclo[2.2.1]heptan-2-yl 4-nitrobenzoate;
decahydronaphthalen-2-yl 4-nitrobenzoate;
decahydronaphthalen-2-yl 4-hydroxybenzoate;
decahydronaphthalen-2-yl 4-hydroxy-3-methyl benzoate;
decahydronaphthalen-2-yl 4-hydroxy-3-methoxybenzoate;
decahydronaphthalen-2-yl 4-amino-3-methyl benzoate;
decahydronaphthalen-2-yl 4-amino-3-methoxybenzoate;
decahydronaphthalen-2-yl 4-fluorobenzoate;
decahydronaphthalen-2-yl 4-fluoro-3-methylbenzoate;
decahydronaphthalen-2-yl 3-methyl-4-nitrobenzoate;
decahydronaphthalen-2-yl 3-methoxy-4-nitrobenzoate;
tricyclo[3.3.1.1^{3,7}]decane-1-carboxylic acid, 3-chloro-, 4-methoxyphenyl ester;
tricyclo[3.3.1.1^{3,7}]decane-1-methanol, 1-β-aminobenzoate);
or tricyclo[3.3.1.1^{3,7}]decane-1-carboxylic acid, 4-hydroxyphenyl ester.

10. (canceled)

11. The compound according to claim 1 selected from the group consisting of the following:
Feroline;
Tschimgine;
Tschimganidine; or
Tschimganine.

12. (canceled)

13. The method of claim 1, wherein the FXR-mediated disease or condition is selected from: cholestasis; colitis; a chronic liver disease selected from primary biliary cirrhosis, primary sclerosing cholangitis, nonalcoholic fatty liver disease, and nonalcoholic steatohepatitis, a gastrointestinal disease selected from inflammatory bowel disease, irritable bowel syndrome, bacterial over-growth, and malabsorption; a cardiovascular disease selected from atherosclerosis, arteriosclerosis, dyslipidemia, hypercholesterolemia, and hypertriglyceridemia; a metabolic disease selected from insulin resistance, hyperglycemia, Type I and Type II diabetes, and obesity; a disorder related to bone formation such as osteoporosis, bone hyperplasia and osteoarthritis; and a kidney disease selected from diabetic nephropathy, focal segmental glomerulosclerosis, chronic glomerulonephritis, interstitial nephritis, acute and chronic renal failure, renal lesions, renal destructive lesions and uremia.

14. The method of claim 9, wherein the FXR-mediated disease or condition is a cancer disease.

15. (canceled)

16. (canceled)

17. A method for lowing triglyceride comprising administering to the mammal a therapeutically effective amount of a compound of claim 9.

18. (canceled)

19. A pharmaceutical composition comprising a pharmaceutical acceptable vehicle and at least one compound selected from the group consisting of the following:
1,7,7-trimethylbicyclo[2.2.1]heptan-2-yl 4-fluoro-3-methoxybenzoate;
1,7,7-trimethylbicyclo[2.2.1]heptan-2-yl 3-bromo-4-fluorobenzoate;
1,7,7-trimethylbicyclo[2.2.1]heptan-2-yl 4-nitrobenzoate;
1,7,7-trimethylbicyclo[2.2.1]heptan-2-yl 3-methyl-4-nitrobenzoate;
cyclooctyl 4-aminobenzoate;
cyclooctyl 4-hydroxy-3-methyl benzoate;
cyclooctyl 4-hydroxy-3-methoxybenzoate;
cyclooctyl 4-hydroxycyclohexanecarboxylate;
1,7,7-trimethylbicyclo[2.2.1]heptan-2-yl 4-hydroxycyclohexanecarboxylate;
1,7,7-trimethylbicyclo[2.2.1]heptan-2-yl 4-aminocyclohexanecarboxylate;
cyclooctyl 4-nitrobenzoate;
bicyclo[2.2.1]heptan-2-yl 4-nitrobenzoate;
decahydronaphthalen-2-yl 4-hydroxybenzoate;
decahydronaphthalen-2-yl 4-hydroxy-3-methyl benzoate;
decahydronaphthalen-2-yl 4-hydroxy-3-methoxybenzoate;
decahydronaphthalen-2-yl 4-amino-3-methylbenzoate;
decahydronaphthalen-2-yl 4-amino-3-methoxybenzoate;
decahydronaphthalen-2-yl 4-fluorobenzoate;
decahydronaphthalen-2-yl 4-fluoro-3-methylbenzoate;
decahydronaphthalen-2-yl 3-methyl-4-nitrobenzoate;
or decahydronaphthalen-2-yl 3-methoxy-4-nitrobenzoate.

20. The method of claim 9, wherein the FXR-mediated disease or condition is an inflammatory disease.

21. The method of claim 9, wherein the FXR-mediated disease or condition is selected from: cholestasis; colitis; a chronic liver disease selected from primary biliary cirrhosis, primary sclerosing cholangitis, nonalcoholic fatty liver disease, and nonalcoholic steatohepatitis, a gastrointestinal disease selected from inflammatory bowel disease, irritable bowel syndrome, bacterial over-growth, and malabsorption; a cardiovascular disease selected from atherosclerosis, arteriosclerosis, dyslipidemia, hypercholesterolemia, and hypertriglyceridemia; a metabolic disease selected from insulin resistance, hyperglycemia, Type I and Type II diabetes, and obesity; a disorder related to bone formation such as osteoporosis, bone hyperplasia and osteoarthritis; and a kidney disease selected from diabetic nephropathy, focal segmental glomerulosclerosis, chronic glomerulonephritis, interstitial nephritis, acute and chronic renal failure, renal lesions, renal destructive lesions and uremia.

22. The method of claim 11, wherein the FXR-mediated disease or condition is selected from: cholestasis; colitis; a chronic liver disease selected from primary biliary cirrhosis, primary sclerosing cholangitis, nonalcoholic fatty liver disease, and nonalcoholic steatohepatitis, a gastrointestinal disease selected from inflammatory bowel disease, irritable bowel syndrome, bacterial over-growth, and malabsorption; a cardiovascular disease selected from atherosclerosis, arteriosclerosis, dyslipidemia, hypercholesterolemia, and hypertriglyceridemia; a metabolic disease selected from insulin resistance, hyperglycemia, Type I and Type II diabetes, and obesity; a disorder related to bone formation such as osteoporosis, bone hyperplasia and osteoarthritis; and a kidney disease selected from diabetic nephropathy, focal segmental glomerulosclerosis, chronic glomerulonephritis, interstitial nephritis, acute and chronic renal failure, renal lesions, renal destructive lesions and uremia.

23. A method for lowing triglyceride comprising administering to the mammal a therapeutically effective amount of a compound of claim 11.
